(19) United States
(12) Patent Application Publication
(10) Pub. No.: US 2024/0252467 A1
(43) Pub. Date: Aug. 1, 2024

(54) TREATMENT OF MENTAL ILLNESS VIA ADMINISTRATION OF BUNTANETAP AND ANALOGUES THEREOF

(71) Applicant: ANNOVIS BIO, INC., Malvern, PA (US)

(72) Inventor: Maria Maccecchini, West Chester, PA (US)

(73) Assignee: ANNOVIS BIO, INC., Malvern, PA (US)

(21) Appl. No.: 18/420,514

(22) Filed: Jan. 23, 2024

Related U.S. Application Data
(60) Provisional application No. 63/440,890, filed on Jan. 24, 2023.

(51) Int. Cl. A61K 31/407; A61P 25/18; A61P 25/22

(52) U.S. Cl. CPC A61K 31/407; A61P 25/18; A61P 25/22

(57) ABSTRACT

The invention relates to methods and pharmaceutical compositions effective for treating, inhibiting, reducing, slowing, or delaying the onset of a mental illness in mammals (e.g., humans) via the administration of an effective amount of a compound selected from the group consisting of Formula (I), Formula (II), Formula (III) or Formula (IV) or pharmaceutically acceptable salts thereof. In certain embodiments, the invention is directed to methods and pharmaceutical compositions of an effective amount of a compound selected from the group consisting of Formula (I), Formula (II), Formula (III) or Formula (IV) or pharmaceutically acceptable salts thereof and an effective amount of an antipsychotic agent, an antidepressant agent, a hallucinogenic agent, and combinations thereof. In certain embodiments, the mental illness is autism, attention deficit-hyperactivity disorder, bipolar disorder, major depressive disorder, or schizophrenia.

This application claims the benefit of U.S. Provisional Application No. 63/440,890, filed on Jan. 24, 2023, hereby incorporated by reference.

### FIELD OF THE INVENTION

The present patent application concerns a method of inhibiting, preventing, or treating mental illness in an animal or a mammal (e.g., human subject or human patient) via administration of buntanetap or related compounds, including their pharmaceutically acceptable salts.

### BACKGROUND OF THE INVENTION

A protein called amyloid beta (“Abeta”) is infamous as a likely contributor to the development of Alzheimer's disease and dementia. Clumps of it, called plaques, are found throughout the brains of people who develop the cognitive symptoms of Alzheimer's disease. For unknown reasons, in a “sick” brain, the level of iron is high, and the synthesis of Abeta and other neurotoxic aggregating proteins is upregulated, leading to overproduction of Abeta and other neurotoxic aggregating proteins in the sick brain. High iron levels increase the translation of neurotoxic proteins, leading to impairment of axonal transport, inflammation, and nerve cell death.

Abeta is a well-known contributor to the development of Alzheimer's disease. Abeta plaques are found throughout the brains of people who develop the cognitive symptoms of Alzheimer's disease.

Five major mental illnesses—autism, attention deficit-hyperactivity disorder, bipolar disorder, major depressive disorder, and schizophrenia—appear to share some commonalities: they all show dysfunctional nerve cell transmission and communication, dysregulation of Abeta protein synthesis similar to neurodegenerative diseases, and they share genetic risk factors, according to an examination of genetic data from more than 60,000 people worldwide (Identification of Risk Loci with Shared Effects on Five Major Psychiatric Disorders: a Genome-Wide Analysis, The Lancet, Vol. 381, Issue 9875, P1371-1379, published online Feb. 28, 2013).

Prior to the present invention, buntanetap, compounds that are similar to buntanetap as described herein, and pharmaceutically acceptable salts and complexes thereof were not used in treatment and prevention of mental illnesses and disorders.

### SUMMARY OF THE INVENTION

It is an object of the invention to provide a treatment for a mental illness or mental disorder in an animal or a mammal (e.g., a human subject or a human patient), including major mental illnesses such as autism, attention deficit-hyperactivity disorder, anxiety, fear, obsessive-compulsive disorder, bipolar disorder, major depressive disorder, and schizophrenia.

It is a further object of the invention to prevent, slow, or delay the development of mental illnesses such as autism, attention deficit-hyperactivity disorder, anxiety, fear, obsessive-compulsive disorder, bipolar disorder, major depressive disorder, and schizophrenia.

In accordance with the above objects and others, the invention is directed in part to a method to inhibit, prevent or treat a mental illness or disorder such as autism, attention deficit-hyperactivity disorder, anxiety, fear, obsessive-compulsive disorder, bipolar disorder, major depressive disorder, and schizophrenia via the administration of buntanetap, compounds that are similar to buntanetap as described herein, and pharmaceutically acceptable salts and complexes thereof. As evidenced by, e.g., the examples of the present invention, buntanetap and pharmaceutically acceptable salts and complexes thereof have utility in inhibiting, preventing and/or treating a mental illness or disorder.

It is a further object of the invention to inhibit, delay or slow the onset of neurological diseases such autism, attention deficit-hyperactivity disorder, anxiety, fear, obsessive-compulsive disorder, bipolar disorder, major depressive disorder and schizophrenia in mammals (e.g., humans) comprising or consisting of administering to the human a therapeutically effective amount of buntanetap, active metabolites of buntanetap, therapeutically effective analogues of buntanetap, compounds that are similar to buntanetap as described herein, pharmaceutically acceptable salts and complexes thereof, and one or more pharmaceutically acceptable excipients. In certain embodiments, buntanetap is administered orally in an amount from about 1 mg to about 120 mg, preferably on a once-a-day basis. In certain embodiments, an effective amount of a compound which is an antipsychotic agent, an antidepressant, and hallucinogen, or any combination thereof are administered concurrently or in the same dosage form.

In certain preferred embodiments, buntanetap is administered in an amount from about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, and numbers in between these numbers, and all further integers to about 120 mg, preferably on a once-a-day basis. In certain preferred embodiments, buntanetap is administered orally in a dose from about, e.g., 2 mg to about 80 mg. In other embodiments, the buntanetap dose is administered intravenously in an amount from about 0.1 to about 25 mg/day. In other preferred embodiments, the buntanetap dose is administered intraperitoneally/intramuscularly (IP/IM) in a dose from about 0.3 mg/day to about 70 mg/day. In certain preferred embodiments, the buntanetap is administered together with an appropriate antidiabetic agent as described further herein in following paragraphs.

In certain embodiments of each of the methods described above, the oral pharmaceutical composition includes from about 1 mg to about 120 mg buntanetap or a pharmaceutically acceptable salt thereof, the IP/IM pharmaceutical composition includes from about 0.3 to about 70 mg buntanetap or a pharmaceutically acceptable salt thereof, and the intravenous (IV) pharmaceutical formulation includes from about 0.1 to about 25 mg buntanetap or a pharmaceutically acceptable salt thereof. Single oral doses of buntanetap above about 120 mg/day and multiple oral doses of up to 240 mg/day are associated with an increased incidence of side effects (e.g., nausea and vomiting

In certain preferred embodiments of the methods described herein, peak plasma circulating levels of buntanetap in humans range, e.g., from about 0.1 ng/mL to about 380 ng/mL, in certain embodiments from about 2 ng/mL to about 20 ng/mL, and more preferably from about 3.7 ng/mL to about 120 ng/mL, and any numbers or integers in between. In certain preferred embodiments, the peak plasma circulating level is reached within about 1 to 3 hours after administration of buntanetap to humans. In certain embodiments, the plasma circulating level of buntanetap is equal to or greater than about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 mg/mL, 15 ng/mL, 16 ng/mL, 17 ng/mL, 18 ng/mL, 19 ng/mL, or 20 ng/mL for at least 9 hours, and preferably for at least 12 hours, after administration of buntanetap to humans in brain. In certain embodiments, the steady-state plasma concentration of buntanetap in brain is at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 ng/mL. In certain embodiments, the half-life of buntanetap in cerebrospinal fluid after administering is about 12 hours, and the half-life of buntanetap in plasma after administering is about 5 hours. In certain embodiments, the administration of buntanetap to humans results in a brain level of buntanetap that range from about 4 to about 10 times the plasma level of buntanetap in those patients. In certain embodiments, the concentration of buntanetap in the brain of humans is from about 8 ng/g to about 3040 ng/g, in certain embodiments from about 30 ng/g to about 960 ng/g.

With respect to each of the methods described above, buntanetap, a pharmaceutically acceptable salt thereof, analogues or similar compounds as described herein may be administered, e.g., orally, parenterally, sublingually, via suppository, nasally, topically, transdermally, or via implant under the skin.

The invention is further directed in part to a method for preventing, treating, inhibiting, delaying, or slowing a neurological disease such as autism, attention deficit-hyperactivity disorder, anxiety, fear, obsessive-compulsive disorder, bipolar disorder, major depressive disorder, and schizophrenia in a human who is at risk of developing such a neurological disease comprising administering to the human a compound selected from the group consisting of Formula (I), Formula (II), Formula (III) and Formula (IV):

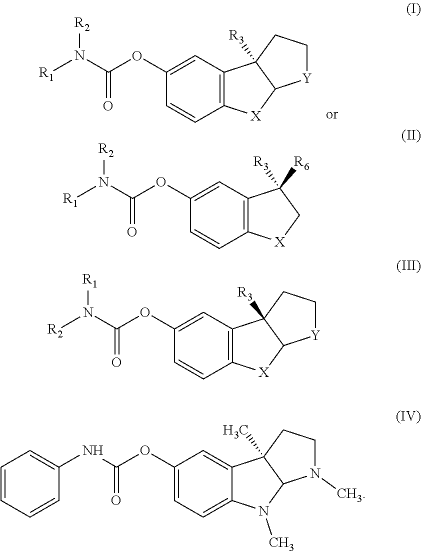

In Formula (I) and Formula (II), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen, C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4. In certain embodiments, the invention is directed to preventing or slowing the ability of melanoma cells to produce their own supply of Abeta with the treatment (administration of buntanetap) as set forth in this paragraph.

In certain embodiments, the compound of Formula (I) and Formula (II) is the substantially pure (+)-enantiomer. In certain embodiments, the compound of Formula (I) is buntanetap or its active metabolites.

In preferred embodiments, the compound is buntanetap of Formula IV as follows:

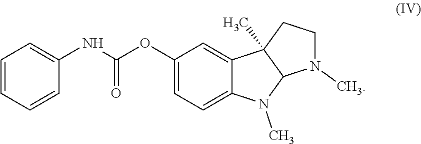

In Formula (III), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl.

The invention is also directed in part a method for preventing or treating anxiety in a human in need thereof comprising administering to the human a therapeutically effective dose of buntanetap or a pharmaceutically acceptable salt thereof. The administration of the therapeutically effective dose of buntanetap or a pharmaceutically acceptable salt thereof to the human may, e.g., prevent the onset of anxiety in the human, completely alleviate in the human or result in at least about 20% reduction in anxiety in the human. The reduction in anxiety may, e.g., be from about 30% to about 100%, from about 40% to about 80%, or from about 40% to about 60%. The reduction in anxiety may, e.g., last for from about 1 minute to about 24 hours, from about 1 minute to about 22 hours, from about 1 minute to about 18 hours, from about 1 min to about 12 hours. The reduction in anxiety may, e.g., last for about 30 minutes, about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours, or about 24 hours.

The invention is further directed in part a method for preventing or treating obsessive-compulsive behavior in a human in need thereof comprising administering to the human a therapeutically effective dose of buntanetap or a pharmaceutically acceptable salt thereof. The administration of the therapeutically effective dose of buntanetap or a pharmaceutically acceptable salt thereof to the human may, e.g., prevent the obsessive-compulsive behavior in the human, completely alleviate the obsessive-compulsive behavior in the human or result in at least about 10% reduction the obsessive-compulsive behavior in the human. The reduction in the obsessive-compulsive behavior may, e.g., be from about 10% to about 90%, from about 40% to about 80%, or from about 20% to about 60%. The reduction in the obsessive-compulsive behavior may, e.g., last for from about 1 minute to about 24 hours, from about 1 minute to about 22 hours, from about 1 minute to about 18 hours, from about 5 min to about 12 hours. The reduction in the obsessive-compulsive behavior may, e.g., last for about 30 minutes, about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours, or about 24 hours.

In addition, the invention is directed in part to a method for preventing or treating fear in a human in need thereof comprising administering to the human a therapeutically effective dose of buntanetap or a pharmaceutically acceptable salt thereof. The administration of the therapeutically effective dose of buntanetap or a pharmaceutically acceptable salt thereof to the human may, e.g., prevent fear in the human, completely alleviate fear in the human or result in at least about 10% reduction in the fear in the human. The reduction in fear may, e.g., be from about 10% to about 90%, from about 40% to about 80%, or from about 20% to about 60%. The reduction in fear may, e.g., last for from about 1 minute to about 24 hours, from about 1 minute to about 22 hours, from about 1 minute to about 18 hours, from about 1 min to about 12 hours. The reduction in fear may, e.g., last for about 30 minutes, about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours, or about 24 hours.

In certain embodiments, the buntanetap or a pharmaceutically acceptable salt thereof is administered (i) orally in an amount from about 1 mg to about 120 mg on a once-a-day basis; (ii) intravenously in an amount from about 0.1 mg to about 25 mg/day; or (ii) intraperitoneally/intramuscularly (IP/IM) in a dose from about 0.3 to about 70 mg/day. The buntanetap or a pharmaceutically acceptable salt thereof is administered, e.g., orally in an amount from about 10 mg to about 80 mg on a once-a-day basis. The peak plasma circulating levels of buntanetap in humans range, e.g., from about 1 ng/mL to about 380 ng/mL.

For the methods for preventing or treating anxiety, obsessive-compulsive disorder or fear, the buntanetap or a pharmaceutically acceptable salt thereof is generally administered orally in an amount from about 1 mg to about 120 mg on a once-a-day basis. The methods for preventing or treating anxiety, obsessive-compulsive disorder or fear in accordance with the present invention therefore include oral administration of an amount from about 1 mg to about 100 mg buntanetap or a pharmaceutically acceptable salt thereof on a once-a-day basis. The methods for preventing or treating anxiety, obsessive-compulsive disorder or fear in accordance with the present invention specifically include administering from about 5 mg to about 80 mg buntanetap or a pharmaceutically acceptable salt thereof on as needed basis up to a maximum daily dose of about 120 mg.

In certain embodiments, the mental illness presents as a disruption in the human genetic code in CACNA1C or CACNB2. In certain embodiments, the mental illness is selected from the group consisting of autism, attention deficit-hyperactivity disorder, bipolar disorder, major depressive disorder, and schizophrenia.

The invention is also directed to a compound selected from the group consisting of Formula (I), Formula (II) and Formula (III) for use in the treatment of a mental illness.

The invention is further directed to buntanetap or a pharmaceutically acceptable salt thereof for use in the treatment of a mental illness.

In addition, the invention is directed to buntanetap or a pharmaceutically acceptable salt thereof for use in the treatment of anxiety.

The invention is further directed to buntanetap or a pharmaceutically acceptable salt thereof for use in the treatment of fear.

The invention is also directed to buntanetap or a pharmaceutically acceptable salt thereof for use in the treatment of obsessive-compulsive behavior.

The invention is further directed to use of a compound selected from the group consisting of Formula (I), Formula (II) and Formula (III) for the manufacture of a medicament for use in the treatment of a mental illness.

The invention is also directed to use of buntanetap or a pharmaceutically acceptable salt thereof for the manufacture of a medicament for use in treatment of a mental illness.

The invention is further directed to use of a compound selected from the group consisting of Formula (I), Formula (II) and Formula (III) for the manufacture of a medicament for treatment of anxiety.

The invention is also directed to use of buntanetap or a pharmaceutically acceptable salt thereof for the manufacture of a medicament for use in the treatment of anxiety.

The invention is further directed to use of a compound selected from the group consisting of Formula (I), Formula (II) and Formula (III) for the manufacture of a medicament for treatment of fear.

The invention is also directed to use of buntanetap or a pharmaceutically acceptable salt thereof for the manufacture of a medicament for use in the treatment of fear.

The invention is further directed to use of a compound selected from the group consisting of Formula (I), Formula (II) and Formula (III) for the manufacture of a medicament for treatment of obsessive-compulsive behavior.

The invention is also directed to use of buntanetap or a pharmaceutically acceptable salt thereof for the manufacture of a medicament for use in the treatment of f obsessive-compulsive behavior.

The invention is further directed in part to a pharmaceutical composition, comprising a therapeutically effective amount of a compound selected from the group consisting of Formula (I), Formula (II) and Formula (III); an effective amount of a compound which is an antipsychotic agent, an antidepressant, and hallucinogen, or any combination thereof; and at least one pharmaceutically acceptable excipient.

In certain preferred embodiments, the compound of Formula (III) is the substantially pure (−)-enantiomer.

In certain preferred embodiments, the compound is buntanetap of Formula (IV),

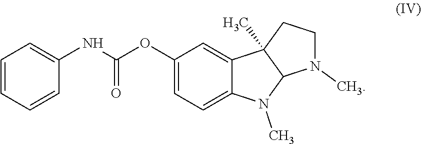 [SMILES: CN1CC[C@]2(C)c3cc(OC(=O)Nc4ccccc4)ccc3N(C)C12]

wherein the compound of Formula (IV) is the substantially pure (+)-enantiomer, or a pharmaceutically acceptable salt thereof.

In certain embodiments, the buntanetap or a pharmaceutically acceptable salt thereof is in an amount from about 1 mg to about 120 mg, and in certain preferred embodiments, the buntanetap or a pharmaceutically acceptable salt thereof is in an amount from about 10 mg to about 120 mg.

In certain embodiments, the antipsychotic agent is a therapeutically effective amount of a typical antipsychotic agent, an atypical antipsychotic agent, a miscellaneous antipsychotic agent, pharmaceutically acceptable salts thereof, and combinations of any of the foregoing. In certain embodiments, the antipsychotic agent is included in a subtherapeutic amount.

In certain embodiments, the pharmaceutical composition includes an antidepressant pharmaceutically acceptable salts thereof, and pharmaceutically acceptable salts thereof, and combinations of any of the foregoing. In certain embodiments, the antidepressant agent or hallucinogenic agent or lithium is included in a subtherapeutic amount.

In certain embodiments, the pharmaceutical composition includes a hallucinogenic agent or pharmaceutically acceptable salts thereof, and combinations of any of the foregoing. In certain embodiments, the hallucinogenic agent is included in a subtherapeutic amount.

In certain embodiments, the pharmaceutical composition includes a therapeutically effective agent selected from Formula (I), Formula (II), Formula (III), or Formula (IV), along with two or more of an antipsychotic agent, an antidepressant agent, lithium, a hallucinogen, pharmaceutically effective salts thereof, or a therapeutically effective combination of an antipsychotic agent, an antidepressant agent, lithium, an hallucinogen, pharmaceutically effective salts thereof. The two or more of an antipsychotic agent, an antidepressant agent, lithium, a hallucinogen, and pharmaceutically effective salts thereof may be included in a therapeutic or subtherapeutic amount.

In certain preferred embodiments, the pharmaceutical composition is an oral dosage form.

In certain preferred embodiments of the pharmaceutical composition, the amount of the compound of formula (I), (II), and (III), or pharmaceutically acceptable salts thereof and the antipsychotic agent, an antidepressant, a hallucinogen, or any combination thereof is effective to treating, inhibiting, reducing, slowing or delaying the symptoms of a mental illness.

In certain embodiments, the mental illness is selected from the group consisting of autism, attention deficit-hyperactivity disorder, bipolar disorder, major depressive disorder, and schizophrenia.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used. In the context of the present application, the term “about” means a value within 20% (±20%) of the value recited immediately after the term “about,” including the value equal to the upper limit (i.e., +20%) and the value equal to the lower limit (i.e., −20%) of this range. For example, the phrase “about 100” encompasses any numeric value that is between 80 and 120, including 80 and 120.

As used herein, the terms “buntanetap” and “posiphen” are used interchangeably to refer to (3aR)-1,3a,8-trimethyl-1,2,3,3a,8,8a-hexahydropyrrolo[2,3-b]indol-5-yl phenylcarbamate or a salt thereof.

As used herein, the term “APP” refers to amyloid precursor protein.

As used herein, the term “Aβ” refers to Abeta or amyloid beta or amyloid-β peptide. For purposes of the present invention, these terms are considered to be synonymous.

For purposes of the present invention, a “buntanetap-type” drug encompasses Formula (I), Formula (II), Formula (III) or Formula (IV).

As used herein, “C9orf72” refers to the C9orf72 protein found in many regions of the brain.

As used herein, the term “neurotoxic aggregating protein” refers to a protein or family of proteins that has neurotoxic effect upon accumulating in a tissue of the brain, such as the brain tissue. Non-limiting examples of neurotoxic aggregating proteins are APP, Aβ, SOD1, SNCA, NAC, TSE amyloid plaque, HTT, Tau, TDP43 and C9orf72.

As used herein, the terms “protein”, “peptide” and “polypeptide” are used interchangeably and refer to a compound comprised of amino acid residues covalently linked by peptide bonds.

By the term “specifically binds,” as used herein, is meant a molecule, such as an antibody or a small molecule, which recognizes and binds to another molecule or feature, but does not substantially recognize or bind other molecules or features in a sample.

The phrase “inhibit” as used herein means to reduce a molecule, a reaction, an interaction, a gene, an mRNA, and/or a protein's expression, stability, function, or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function, and activity, e.g., antagonists.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary, and topical administration.

As used herein, the term “co-administering” refers to a compound of Formula (I), (II), and/or (III) which is administered to a mammal (e.g., a human subject or human patient) together with an appropriate antidiabetic agent so that the two classes of agents provide an overlapping effect. While in certain cases it may be possible to administer the two classes of agents in a single dosage form, it is contemplated that these agents may be separately administered either via the same route of administration or different routes of administration to achieve overlapping effects, taking into account their differing physical/chemical properties (including but not limited to solubility, bioavailability, half-life, metabolism, and clearance/elimination from the body, etc.).

“Pharmaceutically acceptable” refers to a material(s) which are compatible with the activity of the compound useful within the invention and which are physiologically acceptable to the patient (e.g., human) from a pharmacological/toxicological point of view and to the manufacturing pharmaceutical chemist from a physical/chemical point of view regarding composition, formulation, stability, patient acceptance and bioavailability.

“Pharmaceutical acceptable carrier” refers to a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium, and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

An “individual”, “patient”, or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term “treat” or “treating” as used herein means reducing the frequency with which symptoms are experienced by a subject or administering the combination of agents or compounds to reduce the frequency and/or severity with which symptoms are experienced. As used herein, “alleviate” is used interchangeably with the term “treat.” Treating a disease, disorder or condition may or may not include complete eradication or elimination of the symptom. Thus, the term “treating” encompasses preventing and slowing a condition described herein.

The term “therapeutic” as used herein means a treatment and/or prophylaxis of a condition or disease state as described herein.

The term “reduction” as used herein mean a statistically significant reduction.

### BRIEF DESCRIPTION OF THE DRAWINGS

FIG. 1 is a Western blot and a graph showing that buntanetap lowers APP in vitro in a dose-dependent manner in SH-SY-5Y human neuroblastoma cells.

FIG. 2 is a Table, collection of graphs and a Western blot showing that buntanetap treatment of APP/PS1 transgenic AD mice reduced APP and its fragments in hippocampus.

FIG. 3 is a table showing the reduction of APP/Abeta, tau/phospho-tau and αSYN in the spinal fluid of mildly cognitive impaired patients.

FIG. 4 is a diagram showing normal axonal transport in a normal nerve cell (retrograde 0.5 frame/sec), which depicts the normal flow and speed of vesicles carrying D+BDNF across the axon.

FIG. 5 shows abnormal transport in a Down Syndrome nerve cell, which contains high levels of Abeta, tau and αSYN. The transport of BDNF is severely slowed and impaired across the axon.

FIG. 6 demonstrates full recovery of axonal transport in the Down Syndrome nerve cell, when treated with buntanetap for 48 hours.

FIG. 7 shows a summary of the number of marbles covered by sawdust for each group in Example 7.

FIG. 8A to 8E show summaries of the results of the Elevated Plus Maze tests of Example 8.

FIG. 9 shows a summary of the results of the Fear Conditioning Test of Example 9.

### DETAILED DESCRIPTION OF THE INVENTION

The invention is directed in part to a method to inhibit, prevent or treat a mental illness via the administration of buntanetap, compounds that are similar to buntanetap as described herein (e.g., a drug encompassing Formula (I), Formula (II), Formula (III) or Formula (IV)), pharmaceutically acceptable salts and complexes thereof, together with an effective amount of a compound which is an antipsychotic agent, an antidepressant, a hallucinogen, or any combination thereof, and one or more pharmaceutically acceptable excipients.

Mental health and neurological disorders are one of the major groups of noncommunicable diseases (NCDs), which affect people's thoughts, emotions, behaviors, and relationships. They include a diverse range of diseases and conditions such as depression, schizophrenia, dementia, Alzheimer's disease, anxiety, and substance abuse disorders, among many others. An estimated 450-500 million people live with mental conditions worldwide.

Neurological and neuropsychiatric disorders all involve malfunction of the nervous system with impaired neuronal communication. Neurological disorders include Parkinson's disease, Huntington's disease, multiple sclerosis, and Alzheimer's disease. Impaired neuronal communication has been implicated in depression, behavioral problems, posttraumatic stress disorder, attention deficit hyperactivity disorder, and schizophrenia. These disorders can affect social interactions, mood, concentration, memory, and body control. Mental disorders are illnesses that affect the brain, not just the mind and can affect the functioning of an (human) individual; a disorder is a disturbance of the normal physical or mental health of the mind or body. A pathological condition of a body part, an organ, or a system resulting from various causes, such as infection, genetic defect, or environmental stress, and characterized by an identifiable group of signs or symptoms. Because “disease” specifies that the pathological condition be of a body part, people were less likely to use the term “mental illness” before it was understood that mental illness are diseases of the body. The difference between “mental disorder” and “mental illness,” then is one of considering the origin of the condition. The main classes of mental illness include the following.

Neurodevelopmental disorders: This class covers a wide range of problems that usually begin in infancy or childhood, often before the child begins grade school. Examples include autism spectrum disorder, attention-deficit/hyperactivity disorder (ADHD) and learning disorders. Autism spectrum disorder (ASD) is a condition related to brain development (neurological and developmental disorder) that impacts how affects how people interact with others, communicate, learn, and behave. Autistic disorder typically involves language delays, communication challenges, social problems, and unusual interests and behaviors. Although autism can be diagnosed at any age, it is described as a “developmental disorder” because symptoms generally appear in the first two years of life. An estimated 30 to 80 percent of children with autism also meet the criteria for ADHD and, conversely, 20 to 50 percent of children with ADHD for autism. Given the size of the overlap, scientists are beginning to rethink the relationship between the two conditions and to look for common biological roots. The idea that autism and ADHD are intrinsically entwined stems not just from their frequent co-occurrence, but from observations that they share behavioral features. The core diagnostic criteria for the conditions remain distinct in the DSM-5: social communication impairments plus restricted and repetitive behaviors for autism; and inattention or hyperactivity and impulsivity, or a combination, for ADHD. But both conditions can involve delays in language, heightened sensory responses, defiant behavior, problems with regulating emotions and difficulty with planning and with inhibiting behavior. Both also appear in childhood and are more often diagnosed in boys.

Schizophrenia spectrum and other psychotic disorders: Psychotic disorders cause detachment from reality—such as delusions, hallucinations, and disorganized thinking and speech. The most notable example is schizophrenia, although other classes of disorders can be associated with detachment from reality at times. Research suggests that there may be a link between schizophrenia and ASD, as they share common symptoms. Both disorders can vary a lot in their symptoms, but both affect how the brain develops. ASD and schizophrenia may have a genetic link. Some disorders are caused by changes to our chromosomes, the molecules that carry our genetic code. A 2017 study, B. St. Pourcain et al., ASD and Schizophrenia show Distinct Developmental Profiles in Common Genetic Overlap with Population-Based Social Communication Difficulties, Molecular Psychiatry 23, 263-270 (2018), shows that a deletion on chromosome 22 may lead to the development of certain disorders, including ASD and schizophrenia.

Bipolar and related disorders: This class includes disorders with alternating episodes of mania—periods of excessive activity, energy, and excitement—and depression. As with other psychiatric disorders, studies suggest that bipolar disorder may be relatively common among children and adults with autism. Some studies have found that as many as 27 percent of those with autism also have symptoms of bipolar disorder. A. Cardno, et al., Genetic Relationships Between Schizophrenia, Bipolar Disorder and Schizoaffective Disorder, Schizophr Bull., 40(3); 504-515 (2014), states that there is substantial evidence for partial overlap of genetic influences on schizophrenia and bipolar disorder.

Depressive disorders: These include disorders that affect how you feel emotionally, such as the level of sadness and happiness, and they can disrupt your ability to function. Examples include major depressive disorder and premenstrual dysphoric disorder. Depression is when a person experiences depressed mood (feeling sad, irritable, empty) or a loss of pleasure or interest in activities for more than two weeks. Depression can severely impact a person's ability to function and interact with people and society. It is estimated that around 264 million people are affected by depression, or around 5% of adults. Autism and depression often co-occur, and it is well documented that people with an autism diagnosis have a high incidence of co-occurring mental health conditions, and depression is among the most reported. Depression might have same etiological origin as autism and/or share overlapping symptoms, which may be explained by a genetic disposition. B. FC van Heijst, et al., Autism and Depression are Connected: A Report of Two Complimentary Network Studies, Autism. 2020 April; 24(3): 680-692.

Anxiety disorders. Anxiety is an emotion characterized by the anticipation of future danger or misfortune, along with excessive worrying. It can include behavior aimed at avoiding situations that cause anxiety. This class includes generalized anxiety disorder, panic disorder and phobias. Research suggests autistic people are more prone to experiencing anxiety and estimates that up to half of all autistic people experience high levels of anxiety on a regular basis.

Obsessive-compulsive and related disorders. These disorders involve preoccupations or obsessions and repetitive thoughts and actions. Examples include obsessive-compulsive disorder, hoarding disorder and hair-pulling disorder (trichotillomania). There appears to be a link between obsessive-compulsive disorders. See, S. Jacob, et al., Autism Spectrum and Obsessive-compulsive Disorders: OC Behaviours, Phenotypes and Genetics, Autism Res. 2(6):293-311 (209). Therein, it is concluded that autism spectrum disorders (ASD) are a phenotypically and etiologically heterogeneous set of disorders that include obsessive-compulsive disorder (OCD).

Trauma- and stress-related disorders. These are adjustment disorders in which a person has trouble coping during or after a stressful life event. Examples include post-traumatic stress disorder (PTSD) and acute stress disorder.

Dissociative disorders. These are disorders in which your sense of self is disrupted, such as with dissociative identity disorder and dissociative amnesia.

Somatic symptom and related disorders. A person with one of these disorders may have physical symptoms that cause major emotional distress and problems functioning. There may or may not be another diagnosed medical condition associated with these symptoms, but the reaction to the symptoms is not normal. The disorders include somatic symptom disorder, illness anxiety disorder and factitious disorder.

Feeding and eating disorders. These disorders include disturbances related to eating that impact nutrition and health, such as anorexia nervosa and binge-eating disorder.

Elimination disorders. These disorders relate to the inappropriate elimination of urine or stool by accident or on purpose. Bed-wetting (enuresis) is an example.

Sleep-wake disorders. These are disorders of sleep severe enough to require clinical attention, such as insomnia, sleep apnea and restless legs syndrome.

Sexual dysfunctions. These include disorders of sexual response, such as premature ejaculation and female orgasmic disorder.

Gender dysphoria. This refers to the distress that accompanies a person's stated desire to be another gender.

Disruptive, impulse-control and conduct disorders. These disorders include problems with emotional and behavioral self-control, such as kleptomania or intermittent explosive disorder.

Substance-related and addictive disorders. These include problems associated with the excessive use of alcohol, caffeine, tobacco, and drugs. This class also includes gambling disorder.

Personality disorders. A personality disorder involves a lasting pattern of emotional instability and unhealthy behavior that causes problems in your life and relationships. Examples include borderline, antisocial and narcissistic personality disorders.

Paraphilic disorders. These disorders include sexual interest that causes personal distress or impairment or causes potential or actual harm to another person. Examples are sexual sadism disorder, voyeuristic disorder, and pedophilic disorder.

Other mental disorders. This class includes mental disorders that are due to other medical conditions or that don't meet the full criteria for one of the above disorders. In certain embodiments, the mental illness further encompasses acute stress disorder, agoraphobia (anxiety about being in places where escape might not be possible), amnesia, anorexia nervosa, attention deficit/hyperactivity disorder (ADHD), body dysmorphic disorder (preoccupation with a specific body part or parts and believing it or them to be defective), brief psychotic disorder, bulimia nervosa, conversion disorder (deficits in voluntary motor or sensory functions which are not intentionally produced but which cannot be better explained by another health condition, cyclothymic disorder (a milder variant of bipolar disorder), depersonalization disorder (feelings of unreality, that your body does not belong to you, or that you are constantly in a dreamlike state, dissociative identity disorder (DID)), dyspareunia (recurrent or persistent genital pain associate with sexual intercourse not better explained by another medical condition, dysthymic disorder (depressed mood for most of the day on most days for more than two years), erectile disorder, exhibitionism, fetishism, frotteurism (the act of touching or rubbing one's genitals up against another person in a sexual manner without their consent, to derive sexual pleasure or reach orgasm), pathological gambling, generalized anxiety disorder, hypoactive sexual desire disorder, hypochondriasis (preoccupation with fears of having a serious disease based upon misinterpretation of bodily sensations), impotence, intermittent explosive disorder, kleptomania, masochism, major depressive disorder, obsessive-compulsive disorder, orgasmic disorder, pain disorder, panic disorder, pedophilia, phobias, posttraumatic stress disorder, premature ejaculation, pyromania, sadism, schizophrenia, schizoaffective disorder, sexual arousal disorder (female), sexual aversion disorder, shared psychotic, somatization disorder, substance abuse, Tourette syndrome, transvestic fetishism, trichotillomania, vaginismus (recurrent or involuntary spasm of the vaginal muscles that interferes with sexual intercourse not better explained by another medical condition), voyeurism, etc.

The connections between the disorders cited above are merely presented to show that they exist and are not meant to be all-encompassing. For the purposes of the present application, these connections are presented in order to demonstrate that many of these disorders show abnormal patterns of neurotoxic aggregating proteins in the brain, always have abnormal axonal transport and synaptic transmission issues and have at least a partial overlap in etiology, including but not limited to a genetic component.

Serious mental illness is a mental, behavioral, or emotional resulting in serious functional impairment, which substantially interferes with or limits one or more major life activities. Examples of serious mental illness include major depressive disorder, schizophrenia, and bipolar disorder.

Some of the most commonly used classes of prescription psychiatric medications include antidepressants (used to treat depression, anxiety, and sometimes other conditions), anti-anxiety medications, mood-stabilizing medications (most commonly used to treat bipolar disorders), antipsychotic medications (typically used to treat psychotic disorders, such as schizophrenia, bipolar disorders or used with antidepressants to treat depression). Psychotherapy (which involves the patient talking about their condition with a health care provider) is also commonly used. Brain-stimulation treatments are sometimes used for depression and other mental health disorders and are generally reserved for situations in which medications and psychotherapy haven't worked. They include electroconvulsive therapy, repetitive transcranial magnetic stimulation, deep brain stimulation and vagus nerve stimulation.

Antipsychotic medications can reduce or relieve symptoms of psychosis, such as delusions (false beliefs) and hallucinations (seeing or hearing something that is not there). Formerly known as major tranquilizers and neuroleptics, antipsychotic medications are the main class of drugs used to treat people with schizophrenia. They are also used to treat people with psychosis that occurs in bipolar disorder, depression, and Alzheimer's disease. Other uses of antipsychotics include stabilizing moods in bipolar disorder, reducing anxiety in anxiety disorders and reducing tics in Tourette syndrome. The main difference between the first- and second-generation antipsychotics is that the first generation drugs block dopamine and the second generation drugs block dopamine and also affect serotonin levels. Evidence suggests that some of the second-generation drugs have milder movement-related side-effects than the first-generation drugs. Second generation antipsychotics (referred to as “atypical antipsychotics”) include (but are not limited to) risperidone (Risperdal), quetiapine (Seroquel), olanzapine (Zyprexa), ziprasidone (Zeldox), paliperidone (Invega), aripiprazole (Abilify) and clozapine (Clozaril). Although they may not be officially approved for these uses, atypical antipsychotics are sometimes used in the treatment of mood and anxiety disorders, such as bipolar, posttraumatic stress and obsessive-compulsive disorders. Miscellaneous antipsychotic agents are slightly different than the first- and second-generation antipsychotics, having different modes of action to treat mood disorders and schizophrenia. Miscellaneous antipsychotic agents include but are not limited to haloperidol, pimozide, molidone, and loxapine. Phenothiazine antipsychotic agents are thought to work by blocking the action of dopamine in the brain, and include but are not limited to prochlorperazine, chlorpromazine, perphenazine, fluphenazine, thioridazine, and mesoridazine. Another class of antipsychotic agents are thioxanthenes, which are dopamine-2 (D2) receptor antagonists and block the action of dopamine in the brain and include but are not limited to thiothixene. Additional antipyschotic agents useful in the treatments and formulations of the present invention include asenapine, benperidol, zuclopenthixol, flupentixol, fluphenazine, lurasidone, levomepromazine, promazine, pericyazine, caripraine, amisulpride, trifluorperazine, sulpiride, acepromazine, acetophenazine, ziprasidone, iloperidone, and the like.

As reported by the American Psychological Associate, May 2013, Vol 44, No. 5, there are five major mental illnesses—autism, attention deficit-hyperactivity disorder, bipolar disorder, major depressive disorder and schizophrenia—appear to share some common genetic risk factors, according to an examination of genetic data from more than 60,000 people worldwide (The Lancet, online Feb. 28, 2013). Researchers in 19 countries examined the genomes of more than 33,000 individuals with one of the disorders and nearly 28,000 controls. They found four regions of the genetic code where variation was linked to all five disorders. Of particular interest are disruptions in two specific genes. One, CACNA1C, has previously been linked to bipolar disorder and schizophrenia. The other, CACNB2, regulates the flow of calcium in brain cells and is crucial in helping neurons communicate with each other. These researchers believe that the disruption in calcium channel function could be one early pathway that leaves a human vulnerable to developing any of the above-mentioned five disorders.

In certain embodiments, the pharmaceutical composition includes an antipsychotic agent is selected from the group consisting of risperidone, quetiapine, olanzapine, ziprasidone, paliperidone, aripiprazole, clozapine, haloperidol, pimozide, molidone, loxapine, prochlorperazine, chlorpromazine, perphenazine, fluphenazine, thioridazine, mesoridazine, thiothixene, asenapine, benperidol, zuclopenthixol, flupentixol, fluphenazine, lurasidone, levomepromazine, promazine, pericyazine, caripraine, amisulpride, trifluorperazine, sulpiride, acepromazine, acetophenazine, ziprasidone, iloperidone, pharmaceutically acceptable salts thereof, and combinations of any of the foregoing.

In additional embodiments, the pharmaceutical composition includes an antidepressant agent. Many common drugs for depression fall into the following drug classes: selective serotonin reuptake inhibitors (SSRIs) (e.g., citalopram (Cipramil®), dapoxetine (Priligy®), escitalopram (Cipralex®), fluoxetine (Prozac® or Oxactin®), fluvoxamine (Faverin®), paroxetine (Seroxat®), sertraline (Lustral®), vortioxetine (Brintellix®), serotonin-norepinephrine reuptake inhibitors (SNRIs) (e.g., desvenlafaxine, venlafaxine, duloxetine, levomilnacipran); tricyclic antidepressants (TCAs) (e.g., Amitriptyline. Amoxapine. Desipramine (Norpramin®), Doxepin, Imipramine (Tofranil®), Nortriptyline (Pamelor®), protriptyline, trimipramine; tetracyclic antidepressants (amitriptyline, amoxapine. desipramine (Norpramin®), imipramine (Tofranil®), nortriptyline (Pamelor®); dopamine reuptake blockers (e.g., Aplenzin®. Bupropion (Wellbutrin®), bupropion/naltrexone (e.g., Contrave®, Forfivo®XL); 5-HT1A receptor antagonists (e.g., buspirone, flesinoxan, gepirone, flibanserin, nalutzotan, lurasidone, aripiprazole); 5-HT2 receptor antagonists (e.g., clozapine, olanzapine, quetiapine, risperidone and asenapine); 5-HT3 receptor antagonists (e.g., clozapine, olanzapine, quetiapine, risperidone and asenapine); monoamine oxidase inhibitors (MAOIs) (e.g., isocarboxazid (Marplan®), phenelzine (Nardil®), Selegiline (Emsam®), tranylcypromine (Parnate®); noradrenergic antagonists (e.g., propranolol, phentolamine); and atypical antidepressants (which don't fall into these drug classes, and natural supplements, such as St. John's wort; stimulants (e.g., cocaine, MDMA, khat, nicotine, caffeine, amphetamines (including but not limited to amphetamine (Adderall®), methamphetamine, dexamphetamine (Focalin®), methylphenidate (Ritalin®), atomoxetine hydrochloride (Strattera®), lisdexamfetamine dimesylate (Vyvanse®))); and lithium, and immediate release and sustained release versions thereof.

In certain embodiments, the pharmaceutical composition includes a Hallucinogen agent. Hallucinogens (also known as psychedelics) are a group of drugs that change the way people see and feel reality, causing things like hallucinations. Hallucinogens are typically found in plants and fungi or are synthetically produced. They are a large and diverse class of psychoactive drugs that can produce altered states of consciousness characterized by major alterations in thought, mood, and perception as well as other changes. Most hallucinogens can be categorized as either being psychedelics, dissociatives, or deliriants. Many hallucinogens have chemical structures similar to those of natural neurotransmitters, like acetylcholine, serotonin, or catecholamines. Typically, hallucinogens are divided into two types: classic hallucinogens and dissociative drugs. Three principal types of hallucinogenic drugs include psychedelics, dissociatives, and deliriants. Psychedelic drugs work by disrupting how neurotransmitters work in the brain. Drugs in this category are DMT (dimethyltryptamine), peyote, mescaline, magic mushrooms, LSD (D-lysergic acid diethylamide), MDMA (methylenedixoymethamphetamine), psilocybin, psilocin, THC (tetrahydrocannabinol), ketamine, and other therapeutically active cannabinoid molecules contained in marijuana. Mescaline is the main ingredient in the peyote cactus. Psilocybin and psilocin are natural substances found in hallucinogenic mushrooms. MDMA is a synthetic drug derived from amphetamine and works by increasing the activity of the neurotransmitters dopamine, serotonin, and norephinephrine.

Disorders of the mind are rooted in dysfunction of the brain, while neurological disorders interact strongly with psychological and social factors and often cause psychological symptoms. Yet the dominant classifications of mental disorder—the International Classification of Diseases (ICD) and the Diagnostic and Statistical Manual (DSM)1 2—continue to draw a sharp distinction between disorders of the mind, the province of psychiatry, and disorders of the brain, the province of neurology. Some have proposed that psychiatric disorders should be reclassified as disorders of the (central) nervous system. Biological research into mental disorders has been transformed by advances in structural and functional brain imaging, neuropharmacology, and genetics. Meta-analyses have shown that structural brain abnormalities are present in schizophrenia, bipolar affective disorder, recurrent depressive disorder, post-traumatic stress disorder, and obsessive-compulsive disorder. Functional brain imaging has shown that both normal and abnormal emotions have neural representations.

Buntanetap, developed by QR Pharma, Inc. (now Annovis Bio, Inc.), is a small molecule that lowers soluble APP protein levels through a post-transcriptional mechanism. Buntanetap is also known as (+)-phenserine. Buntanetap is the steroisomer of phenserine (−)-N-phenylcarbamoyl eseroline), which reached clinical assessment for AD as an anticholinesterase inhibitor. Phenserine is an AChE inhibitor which has been investigated as being suitable as an agent for therapy for cognitive impairments associated with aging and Alzheimer's disease (U.S. Pat. No. 5,409,948). Due to its high cholinomimetic side effects, phenserine failed in 3 phase 3 clinical studies.

Buntanetap is a selective inhibitor of amyloid precursor protein (APP) production and has potential utility as a disease modifying treatment for AD (Cullen 2006; Utsuki 2006; Lahiri 2007). Buntanetap was discovered at the National Institute on Aging and was selected from a series of structurally related compounds designed for APP specificity with no or minimal acetylcholinesterase inhibitory activity. Buntanetap was shown to reduce APP and consequently beta-amyloid (Aβ) production in relevant preclinical in vitro and in vivo studies. Maccecchini, et al., “Buntanetap as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902, hereby incorporated by reference, reported the results of a study of buntanetap single and multiple ascending dose phase 1 randomized, double blind, placebo-controlled safety, tolerance, pharmacokinetic studies were undertaken in 120 healthy human volunteers to define a dose that was then used in a small non-randomised study of five MCI subjects. Buntanetap doses up to 4×60 mg daily×10 days were well tolerated. In plasma, buntanetap, at all doses, was absorbed rapidly (Tmax=1.2 to 1.7 h) and cleared from the circulation biphasically (terminal half-life of 4.3-4.7 h). Buntanetap proved well tolerated and significantly lowered CSF levels of sAPPα, sAPPβ, t-tau, p-tau, and specific inflammatory markers, and demonstrated a trend to lower CSF Aβ₄₂. Buntanetap's activity is also described in Applicant's U.S. Pat. No. 10,383,851, hereby incorporated by reference. Phase II data for Buntanetap has been published, C. Fang et al, Buntanetap, a Novel Translational Inhibitor of Multiple Neurotoxic Proteins, Proves to Be Safe and Promising in Both Alzheimer's and Parkinson's Patients, J Prev Alzheimers Dis (2022). https://doi.org/10.14283/jpad.2022.84, published 10 Oct. 2022, hereby incorporated by reference in its entirety. This publication reported the results of a Phase 2a Clinical Study which was a double-blind, placebo-controlled, multi-center study of 14 early AD patients and 54 early PD patients. AD patients were given either 80 mg buntanetap or placebo QD. PD patients were given 5 mg, 10 mg, 20 mg, 40 mg, 80 mg buntanetap or placebo QD. The primary endpoint was safety and tolerability; secondary endpoint is pharmacokinetics of buntanetap in plasma. The buntanetap was well tolerated at safe at doses up to 80 mg in both AD and PD patients. Cmax and AUC increased with dose without evidence for a plateau up to 80 mg QD. Biomarker data indicated a trend in lowering levels of neurotoxic proteins and inflammatory factors and improving axonal integrity ad synaptic function in both AD and PD cohorts. Psychometric tests showed statistically significant improvements in ADAS-Cog11 and WAIS coding in AD patients and MDS-UPDRS and WAIS coding in PD patients.

As used herein, the term “buntanetap” refers to (3aR)-1,3a,8-trimethyl-1,2,3,3a,8,8a-hexahydropyrrolo[2,3-b]indol-5-yl phenylcarbamate, with the chemical structure shown in Formula IV below, at a chemical purity of at least 90%, preferably at least 95%, at least 98%, at least 99%, at least 99.5%, at least 99.9% or 100%, having the chemical structural as follows:

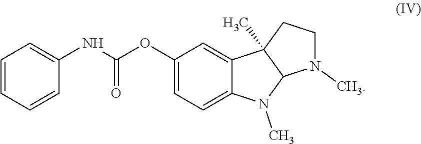 [SMILES: CN1CC[C@]2(C)c3cc(OC(=O)Nc4ccccc4)ccc3N(C)C12]

The term “chemical purity” as applied to (3aR)-1,3a,8-trimethyl-1,2,3,3a,8,8a-hexahydropyrrolo[2,3-b]indol-5-yl phenylcarbamate or a pharmaceutically acceptable salt of Buntanetap means the percent by weight of (3aR)-1,3a,8-trimethyl-1,2,3,3a,8,8a-hexahydropyrrolo[2,3-b]indol-5-yl phenylcarbamate or the pharmaceutically acceptable salt of buntanetap in terms of (3aR)-1,3a,8-trimethyl-1,2,3,3a,8,8a-hexahydropyrrolo[2,3-b]indol-5-yl phenylcarbamate or the pharmaceutically acceptable salt of buntanetap and other chemical impurities, e.g., its (−)-enantiomer, that may be present.

The invention also encompasses active metabolites of buntanetap. Active metabolites have previously been identified and include, for example, “N¹-nor-buntanetap” (which refers to (3aR)-3a,8-dimethyl-1,2,3,3a,8,8a-hexahydropyrrolo[2,3-b]indol-5-yl phenylcarbamate) or a salt thereof; “N⁸-nor-buntanetap” (which refers to (3aR)-1,3a-dimethyl-1,2,3,3a,8,8a-hexahydropyrrolo[2,3-b]indol-5-yl phenylcarbamate) or a salt thereof, and “N¹,N⁸-nor-Buntanetap” (which refers to (3aR)-3a-methyl-1,2,3,3a,8,8a-hexahydropyrrolo[2,3-b]indol-5-yl phenylcarbamate) or a salt thereof.

In other embodiments, the methods of the present invention are practiced using a phenserine or phenserine-like compound, metabolite, enantiomer, or derivative thereof, known to those skilled in the art, such as those described in U.S. Pat. Nos. 5,171,750; 6,410,747; 6,683,105, 7,153,882; 7,786,162; 7,973,057; 8,258,172; 8,546,430; 8,691,864; and 8,853,253, all of which are incorporated by reference in their entireties.

In a broader sense, the invention is directed in part to the administration of a buntanetap-type compound as defined by Formula (I), Formula (II), Formula (III) or Formula (IV) together with an effective amount of a compound which is an antipsychotic agent, an antidepressant, and hallucinogen, or any combination thereof.

The buntanetap-type compounds include compounds having the Formula I or II or III as follows:

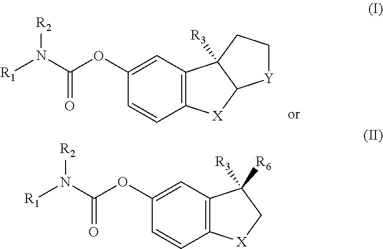

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4, along with an effective amount of an (appropriate) antidiabetic drug.

The chiral center of compounds of Formula I and II is the carbon atom that has R₃ bonded to it. As depicted herein, the (+)-enantiomer has R₃ pointing behind the plane of the page. Although only the (+)-isomer is illustrated to save space, in other embodiments the compound having the Formula I or II can be the (+)-isomer, (−)-isomer, and mixtures of both isomers (e.g., racemic mixtures, including 1:1 racemic mixtures) of all of the compounds encompassed by the invention.

In certain embodiments, the compounds having the Formula I or II have an enantiomeric purity for the (+)-enantiomer of from 55 to 100%, desirably from 75 to 100%, more desirably from 85 to 100%, more desirably from 95 to 100%, and even more desirably 100%.

In certain preferred embodiments, wherein the compound having the Formula I or II is the substantially pure (+)-enantiomer.

In one embodiment, when the compound is Formula I, R₃ is methyl and is X is NCH₃.

In one embodiment, when the compound is Formula I or II, R₃ is not methyl. In particular embodiments, R₃ is a branched or straight chain alkyl or heteroalkyl group of 2, 3, 4, 5, 6, 7, or 8 carbons or substituted or unsubstituted aryl.

In another embodiment, when the compound is Formula I or II, Y is C(H)R₄ or X is O, S, or C(H)R₄.

In another embodiment, when the compound is Formula I, R₃ is methyl, X is NCH₃, and Y is NCH₃. In one embodiment, when the compound is Formula I, R₃ is methyl, X is NCH₃, Y is NCH₃, and R₁ is C₁-C₈ straight chain alkyl or benzyl and R₂ is hydrogen. In one embodiment, when the compound is Formula I, R₃ is methyl, X is NCH₃, Y is NCH₃, and R₁ is substituted or unsubstituted phenyl and R₂ is hydrogen. In one embodiment, when the compound is Formula I, R₃ is methyl, X is NCH₃, Y is NCH₃, and R₁ and R₂ are, independently, methyl or ethyl.

In another embodiment, when the compound is Formula I, R₃ is methyl, X is NCH₃, and Y is O. In one embodiment, when the compound is Formula I, R₃ is methyl, X is NCH₃, Y is O, R₁ is C₁-C₈ straight chain alkyl or benzyl, and R₂ is hydrogen. In one embodiment, when the compound is Formula I, R₃ is methyl, X is NCH₃, Y is O, and R₁ and R₂ are, independently, methyl or ethyl. In one embodiment, when the compound is Formula I, R₃ is methyl, X is NCH₃, Y is O, and R₁ is substituted or unsubstituted phenyl and R₂ is hydrogen.

In another embodiment, when the compound is Formula I, R₃ is methyl, X is NCH₃, and Y is S. In one embodiment, when the compound is Formula I, R₃ is methyl, X is NCH₃, Y is S, R₁ is C₁-C₈ straight chain alkyl or benzyl, and R₂ is hydrogen. In one embodiment, when the compound is Formula I, R₃ is methyl, X is NCH₃, Y is S, and R₁ and R₂ are, independently, methyl or ethyl. In one embodiment, when the compound is Formula I, R₃ is methyl, X is NCH₃, Y is S, R₁ is substituted or unsubstituted phenyl, and R₂ is hydrogen.

In another embodiment, when the compound is Formula I, R₃ is methyl, X is NCH₃, and Y is NR₅. In one embodiment, when the compound is Formula I, R₃ is methyl, X is NCH₃, and Y is NR₅, wherein R₅ is —CH₂CH═CH₂, —CH₂CH₂Ph, benzyl, or hydrogen.

In another embodiment, when the compound has the Formula I, R₃ is methyl, Y is NCH₃, and X is NCH₃, wherein R₄ is benzyl or hydrogen.

In another embodiment, when the compound is Formula I, R₃ is methyl, X is NCH₃, Y is NR₅, wherein each R₄ and R₅ is, independently, hydrogen or benzyl.

In another embodiment, when the compound is Formula I, R₃ is phenyl, X is NCH₃, and Y is NCH₃.

In another embodiment, when the compound is Formula I, R₃ is methyl, and X is NCH₃, and Y is not NH or NHCH₂Ph.

In some embodiments, when the compound is Formula I, R₁ and R₂ are independently, hydrogen, substituted or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ and R₂ are independently, hydrogen or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ is hydrogen, R₂ is unsubstituted aryl, R₃ is methyl, X and Y are independently NR₅, wherein R₅ is independently hydrogen or methyl.

In a certain preferred embodiment, when the compound is Formula I, R₁ is hydrogen, R₂ is phenyl, R₃ is methyl, X is NCH₃, and Y is NCH₃.

In other embodiment, when the compound is Formula I, R₁ is hydrogen, R₂ is phenyl, R₃ is methyl, X is NCH₃, and Y is NH.

In a certain preferred embodiment, when the compound is Formula I, R₁ is hydrogen, R₂ is phenyl, R₃ is methyl, X is NH, and Y is NCH₃.

In a certain preferred embodiment, when the compound is Formula I, R₁ is hydrogen, R₂ is phenyl, R₃ is methyl, X is NH, and Y is NH.

In another embodiment, when the compound is Formula II, R₃ is methyl, X is C(H)CH₃, and R₆ is (CH₂)₂R₇, where R₇ is a substituted or unsubstituted amino group.

In a certain preferred embodiment, when the compound is Formula II, R₃ is methyl, X is NCH₃, and R₆ is (CH₂)₂R₇, where R₇ is a substituted or unsubstituted amino group.

In a certain preferred embodiment, wherein the compound having the Formula II is the substantially pure (+)-enantiomer.

The invention also relates to the use of a compound having the Formula (III) as follows:

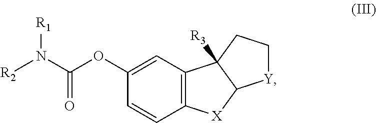

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl.

As depicted herein, the (−)-enantiomer has R₃ pointing in front of the plane of the page. Although only the (−)-isomer is illustrated to save space, in other embodiments the compound having the Formula (III) can be the (+)-isomer, (−)-isomer, and mixtures of both isomers (e.g., racemic mixtures, including 1:1 racemic mixtures) of all of the compounds encompassed by the invention.

In a certain preferred embodiment, wherein the compound having the Formula (III) is the substantially pure (−)-enantiomer.

In one embodiment, when the compound is Formula (III), X is NR₅, wherein R₅ is aralkyl.

In one embodiment, when the compound is Formula (III), X and Y are NR₅, wherein R₅ is aralkyl.

In one embodiment, when the compound is Formula (III), wherein X is NR₅, wherein R₅ is aralkyl, and Y is NR₅, wherein R₅ is branched or straight chain C₁₋₈ alkyl or heteroalkyl.

In one embodiment, when the compound is Formula (III), wherein R₁ is branched or straight chain C₁-C₈ alkyl, aralkyl or aryl, R₂ is hydrogen, branched or straight chain C₁-C₈ alkyl, substituted or unsubstituted aryl, or aralkyl; Y is NR₅, wherein R₅ is aralkyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NR₅ where R₅ is benzyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NR₅ where R₅ is benzyl; and X is NR₅, wherein R₅ is hydrogen.

In one embodiment, when the compound is Formula (III), wherein R₁ is para-halophenyl; Y is NCH₃; and X is NR₅, wherein R₅ is alkyl or aralkyl, wherein R₁ is not para-phenyl bromophenyl when R₅ is benzyl.

In one embodiment, when the compound is Formula (III), wherein R₁ is para-isopropyl phenyl; R₂ is hydrogen; R₃ is methyl; Y is NR₅ where R₅ is benzyl; and X is NR₅, wherein R₅ is hydrogen.

Encompassed in the formulations of the invention are the (+)-isomer, (−)-isomer, and mixtures of both isomers (e.g., racemic 1:1 mixtures) of all of the compounds of the invention unless such compounds are specifically excluded.

Variables, such as R₁-R₇, n, X and Y throughout the application are the same variables as previously defined unless stated to the contrary.

The term “alkyl” as used herein refers to a branched or unbranched saturated hydrocarbon group of 1 to 4, 1 to 8, or 1 to 20 carbon atoms, such as methyl, ethyl, n-propyl, isopropyl, n-butyl, isobutyl, t-butyl, pentyl, hexyl, heptyl, octyl, and the like. Examples of cycloalkyl groups include cyclopentyl and cyclohexyl.

The term “alkenyl” as used herein refers to a hydrocarbon group of 2 to 4, 2 to 8, or 2 to 20 carbon atoms and structural formula containing a carbon-carbon double bond.

The term “alkynyl” as used herein refers to a hydrocarbon group of 2 to 4, 2 to 8, or 2 to 20 carbon atoms and a structural formula containing a carbon-carbon triple bond.

The term “aryl” is defined as any carbon-based aromatic group including, but not limited to, phenyl, benzene, naphthalene, anthracene, phenanthrene, pyrene, and benzo[a]pyrene, etc.

The term “substituted aryl” is defined as an aryl group having at least one group attached to the aryl group that is not hydrogen. Examples of groups that can be attached to the aryl group include, but are not limited to, alkyl, alkynyl, alkenyl, aryl, heterocyclic, halide, nitro, amino, ester, ketone, aldehyde, hydroxy, carboxylic acid, alkoxy, cyano, alkoxy, thioalkyl, haloalkyl, hydroxyalkyl, alkylamino, diakylamino, or acyl. In various embodiments, a substituent is bound to carbon 2, 3, 4, 5, or 6 of one of these moieties. Examples of alkoxy substituents include, but are not limited to, methoxy, ethoxy, and isopropoxy groups. Examples of acyl substituents include acetyl and benzoyl groups.

The term “aralkyl” is defined as an aryl group having an alkyl, alkynyl, or alkenyl group attached to the aryl group. An example of an aralkyl group is a benzyl group.

The term “heteroaryl” is defined as an aryl group that has at least one heteroatom such as nitrogen, sulfur, or oxygen incorporated within the ring of the aryl group.

The term “heteroalkyl” is defined as an alkyl group that has at least one heteroatom, such as nitrogen, sulfur, oxygen, or phosphate, incorporated within the alkyl group or attached to the alkyl group.

The compounds described herein may form salts with acids or bases, and such salts are included in the present invention. In one embodiment, the salts are pharmaceutically acceptable salts. The term “salts” embraces addition salts of free acids or free bases that are compounds of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification, or formulation of compounds of the invention.

Examples of the pharmaceutically acceptable salt of buntanetap include acid addition salts prepared from a suitable acid. The suitable acid can be hydrobromic acid, hydrochloric acid, hydroiodic acid, sulfuric acid, carbonic acid, nitric acid, phosphoric acid, tetrafluoroboronic acid, perchloric acid, 1-hydroxy-2-naphthoic acid, 2,2-dichloroacetic acid, methanesulfonic acid, trifluoromethanesulfonic acid, 2-hydroxyethanesulfonic acid, benzenesulfonic acid, p-toluenesulfonic acid, cyclohexylaminosulfonic acid, 2-oxoglutaric acid, 4-acetamidobenzoic acid, acetic acid, phenylacetic acid, propionic acid, formic acid, succinic acid, glycolic acid, gluconic acid, malic acid, lactic acid, tartaric acid, citric acid, glucuronic acid, ascorbic acid, maleic acid, fumaric acid, pyruvic acid, aspartic acid, glutamic acid, benzoic acid, 4-hydroxybenzoic acid, anthranilic acid, 4-hydroxybenzoic acid, mandelic acid, pamoic acid, pantothenic acid, sulfanilic acid, stearic acid, alginic acid, p-hydroxybutyric acid, salicylic acid, galactaric acid and galacturonic acid. Preferably, the pharmaceutically acceptable salt is buntanetap tartrate, i.e., the acid addition salt of tartaric acid.

Suitable pharmaceutically acceptable base addition salts of compounds of the invention include, for example, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N methylglucamine) and procaine. Examples of pharmaceutically unacceptable base addition salts include lithium salts and cyanate salts. All of these salts may be prepared from the corresponding compound by reacting, for example, the appropriate acid or base.

### Administration and Dosing

In the methods of the invention, buntanetap, its analogs, metabolites, or a pharmaceutically acceptable salt thereof (with or without an antipsychotic, an antidepressant, a hallucinogenic or any combination thereof) can be administered parenterally or enterally. Examples of the route of administration of buntanetap, or an analog, metabolite, (and an antipsychotic, an antidepressant, a hallucinogenic or any combination thereof) or pharmaceutically acceptable salts thereof are intravenous, intraocular, intramuscular, subcutaneous, topical, oral, sublingual, and buccal. Preferably, for purposes of the present invention, buntanetap (with or without one of an antipsychotic, an antidepressant, a hallucinogenic or any combination thereof) is administered orally.

In the present invention, buntanetap, or a pharmaceutically acceptable salt of buntanetap, can be administered once, twice, three times, or four times daily. Buntanetap is preferably administered on a once-a-day basis. Depending on the route of administration, buntanetap is administered in different dose ranges.

In certain embodiments (buntanetap) is administered orally in an amount from about 1 mg to about 120 mg, preferably on a once-a-day basis. In certain preferred embodiments, buntanetap is administered in an amount from about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, and numbers in between these numbers, and all further integers to about 120 mg, preferably on a once a day basis. In certain preferred embodiments, buntanetap is administered orally in a dose from about 10 mg to about 80 mg. In other embodiments, the buntanetap dose is administered intravenously in an amount from about 0.1 to about 25 mg/day. In other preferred embodiments, the buntanetap dose is administered intraperitoneally/intramuscularly (IP/IM) in a dose from about 0.3 to about 70 mg/day.

Doses for humans believed to be efficacious and safe are set forth in Table 1 below for oral, IP/IM, and IV routes of administration:

TABLE 1

| ORAL Human | IP/IM Human | IV Human |
|---|---|---|
| Efficacious and | Efficacious and | Efficacious and |
| Safe Dose mg/day | Safe Dose mg/day | Safe Dose mg/day |
| | | 0.1 |
| | 0.3 | 0.3 |
| 1.0 | 1.0 | 1.0 |
| 4.9 | 4.9 | 4.9 |
| 10.0 | 10.0 | 10.0 |
| 11.2 | 11.2 | 11.2 |
| 14.7 | 14.7 | 14.7 |
| 16.1 | 16.1 | 16.1 |
| 20.3 | 20.3 | 20.3 |
| 23.8 | 23.8 | 23.8 |
| 39.9 | 39.9 | |
| 70.0 | 70.0 | |
| 79.8 | | |
| 119.7 | | |
| 1 to 120 | 0.3 to 70 | 0.1 to 25 |

In certain embodiments of each of the methods of the present invention as described above, the oral pharmaceutical composition includes from about 1 mg to about 120 mg buntanetap or a pharmaceutically acceptable salt thereof, the IP/IM pharmaceutical composition includes from about 0.3 to about 70 mg buntanetap or a pharmaceutically acceptable salt thereof, and the intravenous (IV) pharmaceutical formulation includes from about 0.1 to about 25 mg buntanetap or a pharmaceutically acceptable salt thereof.

In general, the dose of buntanetap preferred to be administered to animals or human patients is a tolerable dose, i.e., a dose that does not cause untoward side effects in a majority of human patient, which dose is also effective for prophylactic treatment of the healthy human(s) with respect to, e.g., neurodegenerative diseases, cancer, cardiovascular homeostasis, diseases or conditions of vital organs, cardiovascular disease, and the like.

In certain preferred embodiments of the methods described herein, peak plasma circulating levels of buntanetap in humans range, e.g., from about 1 ng/mL to about 380 ng/mL, in certain embodiments from about 2 ng/mL to about 20 ng/mL, and more preferably from about 3.7 ng/mL to about 120 ng/mL. In certain preferred embodiments, the peak plasma circulating level is reached within about 6 hours after administration of buntanetap to humans. In certain embodiments, the peak plasma circulating level is reached within about 3 hours after administration of buntanetap to the humans. In certain embodiments, the plasma circulating level of buntanetap is equal to or greater than about 0.1 ng/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 mg/mL, 15 ng/mL, 16 ng/mL, 17 ng/mL, 18 ng/mL, 19 ng/mL, or 20 ng/mL for at least 9 hours, and preferably for at least 12 hours, after administration of buntanetap to humans. In certain embodiments, the steady-state plasma concentration of buntanetap is at least about 0.01, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 4647, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 11, 112, 113, 114, 115, 116, 117, 118, 119, 120, 121, 122, 123, 124, 125, 126, 127, 128, 129 or 130 ng/mL, and numbers in between these numbers. In certain embodiments, the half-life of buntanetap in cerebrospinal fluid after administering is about 12 hours, and the half-life of buntanetap in plasma after administering is about 5 hours. In certain embodiments, the administration of Buntanetap to humans results in a brain level of buntanetap that range from about 4 to about 10 times the plasma level of buntanetap in those patients. In certain embodiments, the concentration of buntanetap in the brain of humans is from about 8 ng/g to about 3040 ng/g, in certain embodiments from about 30 ng/g to about 960 ng/g.

Table 2 provides plasma levels for humans and brain levels for humans as calculated and extrapolated from animal (mice data):

TABLE 2

| | | | Plasma | | |
|---|---|---|---|---|---|
| | | Plasma | levels in | Brain levels | Brain levels |
| Dose | mg dose | levels in | human | in mice | in humans |
| mg/kg | for 70 kg | mice n/ml | ng/ml | ng/gram | ng/gram |
| 0.014 | 1.0 | 0 | 1 | 0 | 8 |
| 0.07 | 4.9 | 0 | 2 | 0 | 16 |
| 0.143 | 10.0 | 0 | 3.7 | 0 | 29.6 |
| 0.16 | 11.2 | 0 | 5.7 | 0 | 45.6 |
| 0.21 | 14.7 | 0 | 11.5 | 0 | 92 |
| 0.23 | 16.1 | 0 | 12.9 | 0 | 103.2 |
| 0.29 | 20.3 | 0 | 14 | 0 | 112 |
| 0.34 | 23.8 | 0 | 20 | 0 | 160 |
| 0.57 | 39.9 | 0 | 42 | 0 | 336 |
| 1 | 70.0 | 2 | 90 | 16 | 720 |
| 1.14 | 79.8 | 2.1 | 120 | 16.8 | 960 |
| 1.71 | 119.7 | 0 | 200 | 0 | 1600 |
| 2.13 | 149.1 | 3.9 | 240 | 31.2 | 1920 |
| 2.29 | 160.3 | 0 | 380 | 0 | 3040 |
| 3 | | 6 | | 48 | |
| 5 | | 30 | | 80 | |
| 10 | | 20 | | 160 | |
| 20 | | 50 | | 400 | |
| 30 | | 100 | | 800 | |
| 40 | | 150 | | 1200 | |
| 50 | | 210 | | 1680 | |
| 60 | | 300 | | 2400 | |
| 80 | | 600 | | 4800 | |
| 100 | | 1000 | | 8000 | |

The therapeutic agent(s) used as the buntanetap-type drug are preferably dosed in therapeutically effective amounts known to those skilled in the art. In certain embodiments, the therapeutically effective amount is an amount that yields a maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. One skilled in the clinical and pharmacological arts will be able to determine a therapeutically effective amount through routine experimentation, namely by monitoring a subject's response to administration of the agent and adjusting the dosage accordingly. In certain embodiments, the buntanetap-type drug is administered via a preferred appropriate route of administration (e.g., oral, subcutaneous, intravenous, intramuscular).

### Pharmaceutical Compositions and Therapies

Administration of compounds useful within the invention may be achieved in a number of different ways, using methods known in the art. The therapeutic and prophylactic methods of the invention thus encompass the use of pharmaceutical compositions comprising the compounds useful within the invention to practice the methods of the invention.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Typically, buntanetap dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 μg to about 10 mg per kilogram of body weight of the animal. More preferably, the dosage will vary from about 3 μg to about 30 mg per kilogram of body weight of the animal.

Pharmaceutical compositions that are useful in the methods of the invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses of, e.g., the buntanetap and the (optional) additional drug, e.g., an antipsychotic, an antidepressant, a hallucinogenic or any combination thereof). As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The composition of the invention may consist of the active ingredient alone, in a form suitable for administration to a (human) subject or patient, or the composition may comprise at least one active ingredient and one or more pharmaceutically acceptable excipients.

In one embodiment, the compositions of the invention are formulated using one or more pharmaceutically acceptable carriers. Pharmaceutically acceptable carriers that are useful, include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The carrier may be a solvent or dispersion medium containing, for example, water, ethanol, polyol (for example, glycerol, propylene glycol, and liquid polyethylene glycol, and the like), suitable mixtures thereof, and vegetable oils.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an antioxidant and a chelating agent that inhibits the degradation of the compound. Preferred antioxidants for some compounds are BHT, BHA, alpha-tocopherol, and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing, or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Controlled- or sustained-release formulations of a composition of the invention may be made using conventional technology, in addition to the disclosure set forth elsewhere herein. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans-, and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

For oral administration, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more inert, non-toxic pharmaceutically excipients. Such excipients include, for example an inert diluent such as lactose; granulating and disintegrating agents such as cornstarch; binding agents such as starch; and lubricating agents such as magnesium stearate. The oral compositions of the invention in the form of tablets or capsules prepared by conventional means with pharmaceutically acceptable excipients such as binding agents; fillers; lubricants; disintegrates; or wetting agents.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. For oral administration, if desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400).

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid preparation for oral administration may be in the form of solutions, syrups, or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters, or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications.

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### DETAILED DESCRIPTION OF PREFERRED EMBODIMENTS

The following examples further illustrate aspects of the present invention. They are provided for the purpose of illustration only, and the invention is not limited to these examples, but rather encompasses all variations that are evident as a result of the teachings provided herein.

### Example 1

Three Phase I clinical studies have established buntanetap's safety. The pharmacokinetic analyses have demonstrated that the small lipophilic molecule readily enters the brain where its concentration is about 8 times higher than in plasma. Importantly, buntanetap normalized levels of APP, Tau, and αSYN in the cerebrospinal fluid (CSF) of MCI subjects at a dose of 4×60 mg/day. Buntanetap had a >12 h half-life in CSF of MCI subjects, and its effect in lowering these neurotoxic proteins and inflammation extended throughout the 12 h sampling period after the last dose (Maccecchini, et al., “Buntanetap (buntanetap) as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902). Therefore, we conclude that a much lower single daily buntanetap dose would be effective in the proposed study. In fact, much lower doses were studied in a double Alzheimer/Parkinson phase 2 study that was conducted and completed in 2021 (effective IND #72,654). The double phase 2 clinical trial recruited 14 AD (Alzheimer's disease) and 54 patients and treated them over 25 with a once daily dose of buntanetap. The 14 AD patients receive either 80 mg QD or placebo, whereas the 54 PD (Parkinson's disease) patients received 5, 10 20, 40 80 mg QD or placebo. In a nutshell the data shows that in AD and in PD patients (a) buntanetap crossed the blood brain barrier, (b) reduced neurotoxic protein biomarkers, (c) reduced inflammatory markers, (d) improved axonal and synaptic function, and most importantly improved the affected function in both patient populations. In AD patients, buntanetap improved cognition as measured by ADAS-Cog11 and WAIS coding speed (achieving statistical significance versus baseline at 80 mg dose but not placebo). In PD patients it improved at all doses motor function as measured by MDS-UPDRS (Part II, III, IV and total) with the maximum improvement for 10 & 20 mg and improved WAIS speed and accuracy (achieving statistical significance versus placebo in the 5 mg, 20 mg and 80 mg dose arms [p<0.05] of the broader study population [n=54] with the total for all doses also reporting statistically significant improvement [p<0.001]). The data demonstrates the potential benefits of reducing the overexpression of neurotoxic aggregating proteins on inflammation, axonal and synaptic function, and cognitive and functional health. We expect a larger sample population will allow buntanetap to fully demonstrate statistically significant cognitive and functional improvement resulting from the normalization of toxic protein levels in the ongoing phase 3 Parkinson's study and phase 2/3 Alzheimer's study.

Buntanetap's effect on neurotoxic proteins: the drug lowers levels of APP in vitro in neuroblastoma cells (Mikillineni et al: Parkinson's Disease; Volume 2012, Article ID 142372, 13 pages. The Anticholinesterase Phenserine and Its Enantiomer Buntanetap as 5′ Untranslated-Region-Directed Translation Blockers of the Parkinson's Alpha Synuclein Expression). It also lowers levels of APP and all its fragments in APP/PS1 transgenic mice (A. F. Teich, et al., Alzheimer's & Dementia: Translational Research & Clinical Interventions 4 (2018) 37-45).

Buntanetap also lowers tau in vitro (Peter Davies Laboratory, Hofstra University, unpublished observation) and in vivo in Human tau mice (Peter Davies Laboratory, Hofstra University, unpublished observation). Buntanetap further lowers αSYN in vitro in neuroblastoma cells and in vivo in transgenic Parkinson's animals in the brain and in the gut (Kuo et. al. Am J Neurodegener Dis 2019; 8(1):1-15 www.AJND.us/ISSN:216 591X/AJND0086080: Translational inhibition of α-synuclein by Buntanetap (buntanetap) normalizes distal colon motility in transgenic Parkinson mice).

Buntanetap's reversal of the neurotoxic cascade: buntanetap's mechanism of action is related to APP, Tau, and αSYN expression being regulated by RP1 and by iron and the way these proteins contribute to neurodegeneration by accumulating as toxic aggregates that impair axonal transport and synaptic transmission, causing inflammation, and, finally, leading to nerve cell death (as described previously). By reducing APP, Tau, and αSYN levels, buntanetap treatment prevented this toxic cascade. In support of this hypothesis, it has been shown that buntanetap: Normalized anterograde and retrograde vesicle transport in fully differentiated Down syndrome nerve cells [W. Mobley; USCD]; Normalized impaired synaptic transmission in rat striatum after traumatic brain injury (TBI) [M-F Chesselet; UCLA] and hippocampus of APP/PS1 tg mice; lowered inflammation in human CSF of MCI subjects and in the rat brain after TBI; protected nerve cells in rat substantia Ingra after TBI and in a rat acute glaucoma model (J Sundstrom; Hershey Medical School).

The AD field has been dominated by approaches to prevent APP processing or remove Aβ in one of its many forms. These are downstream targets; buntanetap prevents the translational synthesis of the two main proteins involved in AD—APP and tau—and hence should remove all the downstream consequences produced by these proteins. Similarly, the PD field mostly focuses on inhibiting accumulation of αSYN aggregates and the effect of other proteins in this pathway, including LRRK or Parkin. Again, buntanetap prevents the synthesis of αSYN and thus it should stop the pathological cascade at the first step. Our data indicate that by normalizing the levels APP/Aβ, Tau/phospho-Tau, and αSYN, buntanetap normalizes axonal transport, lowers inflammation, and protects nerve cells from dying. (Mobley 2020, submitted for publication; Chesselet 2020, submitted for publication).

### Example 2

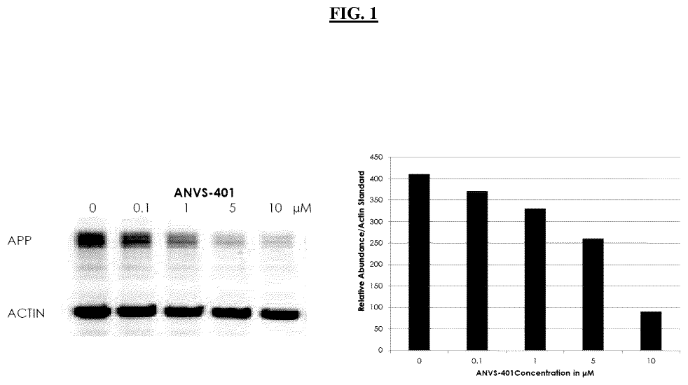

FIG. 1, APP in vitro. shows that buntanetap lowers APP in vitro in a dose-dependent manner in SH-SY-5Y human neuroblastoma cells. On the left is a Western blot showing buntanetap inhibition of APP in relation to Actin standard at concentrations of 0, 0.1, 1, 5 and 10 μM and a graph showing the same data plotted for statistical analysis purposes.

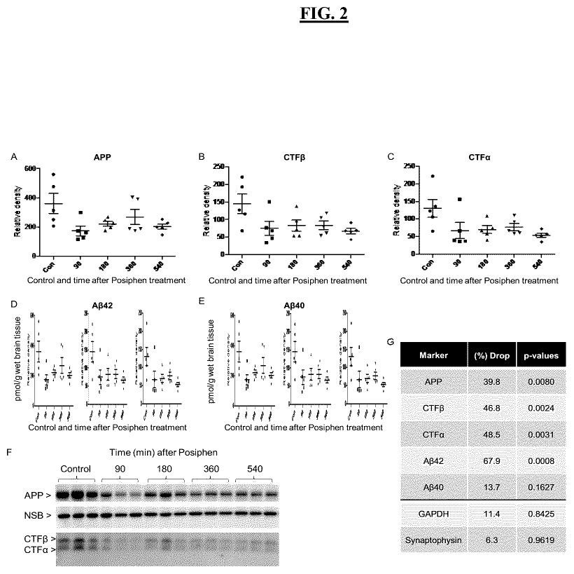

FIG. 2, APP in vivo. This study was conducted to demonstrate the effect of buntanetap in inhibiting the translation of APP and its fragments in an AD model in vivo. The Table in FIG. 2 shows that buntanetap treatment of APP/PS1 transgenic AD mice reduced APP and its fragments in hippocampus. GAPDH and Synaptophysin were loading controls. FIG. 2 also includes a collection of graphs showing relative density of APP plotted against control and time after buntanetap treatment; relative density of CTFβ plotted against control and time after buntanetap treatment; relative density of CTFα plotted against control and time after buntanetap treatment; Aβ42 levels in brain tissue plotted against control and time after buntanetap treatment; and Aβ40 levels in brain tissue plotted against control and time after buntanetap treatment. Finally, FIG. 2 also includes a Western blot showing levels of APP, NSB, CTFβ, and CTFα after buntanetap treatment over time (minutes). In APP/PS1 mice expressing human mutations associated with familial AD, the data show that buntanetap treatment reduced APP and all related peptides in hippocampus for at least 9 hours after the last dose.

### Example 3

FIG. 3 is a study of subjects with mild cognitive impairment (MCI) for early proof of mechanism (POM) using a well-tolerated dose of buntanetap. Before and after 10 days of buntanetap administration to the MCI subjects, plasma, and cerebrospinal fluid (CSF) samples were obtained for analysis of levels of secreted (s) APPα and APPβ, and Aβ42, Tau (total and phosphorylated), and inflammatory markers. FIG. 3 shows the reduction of APP/Aβ, tau/phosphor-tau and αSYN in the spinal fluid of mildly cognitive impaired patients. In this study, buntanetap normalized these aggregating proteins in CSF of MCI subjects in accordance with the data seen in animals.

### Example 4

Table 3 below provides dose that are useful in humans and mice. The doses are provided in mg/kg; mg dose for a 70 kg human; plasma levels in mice and humans (ng/mL); plasma levels in humans (nM); brain levels in mice and humans (ng/gram); oral human efficacious and safe dose (mg/day); IP/IP human efficacious and safe dose (mg/day); and IV human efficacious and safe dose (mg/day). It is believed that efficacious IP/IM dosing is in the middle of these ranges (e.g., about 60 mg/day based on the oral dose; about 35 mg/day based on the IP/IP human dose; and about 12.5 mg/day based on the IV dose). Between about 120 mg/day and 200 mg/day, buntanetap turns toxic. The dose of 1 nM buntanetap was found to be efficacious, as was the (extrapolated) plasma level of about 10 to about 10,000 nM in tissue culture.

TABLE 3

| | | | | | | | ORAL | IP/IP | |
|---|---|---|---|---|---|---|---|---|---|
| | | | Plasma | Plasma | Brain | Brain | Human | Human | IV Human |
| | mg | Plasma | levels | levels | levels | levels | Efficacious | Efficacious | Efficacious |
| | dose | levels | in | in | in | in | and Safe | and Safe | and Safe |
| Dose | for | in mice | human | humans | mice | humans | Dose | Dose | Dose |
| mg/kg | 70 kg | ng/ml | ng/mL | nM | ng/gram | ng/gram | mg/day | mg/day | mg/day |
| | | | | | | | | | 0.1 |
| | | | | | | | | 0.3 | 0.3 |
| 0.014 | 1.0 | 0 | 1 | 3 | 0 | 8 | 1.0 | 1.0 | 1.0 |
| 0.07 | 4.9 | 0 | 2 | 6 | 0 | 16 | 4.9 | 4.9 | 4.9 |
| 0.143 | 10.0 | 0 | 3.7 | 10 | 0 | 29.6 | 10.0 | 10.0 | 10.0 |
| 0.16 | 11.2 | 0 | 5.7 | 17 | 0 | 45.6 | 11.2 | 11.2 | 11.2 |
| 0.21 | 14.7 | 0 | 11.5 | 32 | 0 | 92 | 14.7 | 14.7 | 14.7 |
| 0.23 | 16.1 | 0 | 12.9 | 38 | 0 | 103.2 | 16.1 | 16.1 | 16.1 |
| 0.29 | 20.3 | 0 | 14 | 41 | 0 | 112 | 20.3 | 20.3 | 20.3 |
| 0.34 | 23.8 | 0 | 20 | 58 | 0 | 160 | 23.8 | 23.8 | 23.8 |
| 0.57 | 39.9 | 0 | 42 | 123 | 0 | 336 | 39.9 | 39.9 | |
| 1 | 70.0 | 2 | 90 | 264 | 16 | 720 | 70.0 | 70.0 | |
| 1.14 | 79.8 | 2.1 | 120 | 352 | 16.8 | 960 | 79.8 | | |
| 1.71 | 119.7 | 0 | 200 | | 0 | 1600 | 119.7 | | |
| 2.13 | 149.1 | 3.9 | 240 | | 31.2 | 1920 | | | |
| 2.29 | 160.3 | 0 | 380 | | 0 | 3040 | 1 to 120 | 0.3 to 70 | 0.1 to 25 |
| 3 | | 6 | | | 48 | | | | |
| 5 | | 10 | | | 80 | | | | |
| 10 | | 20 | | | 160 | | | | |
| 20 | | 50 | | | 400 | | | | |
| 30 | | 100 | | | 800 | | | | |
| 40 | | 150 | | | 1200 | | | | |
| 50 | | 210 | | | 1680 | | | | |
| 60 | | 300 | | | 2400 | | | | |
| 80 | | 600 | | | 4800 | | | | |
| 100 | | 1000 | | | 8000 | | | | |

### Example 5—Neurodegeneration is an Axonal Transport Disease

In Example 5, it is shown that axonal transport disruption is linked to human neurological conditions (Nature Review, September 2019). Axonal transport it responsible for a number of human neurological conditions, including but not limited to neurotransmitters GABA (anxiety), Ach (cognition), dopamine (movement) and serotonin (mood); neurotrophic factors (NGF, BDBF); and all communication within and between nerve cells. Thus, chronic and acute brain insults lead to high levels of neurotoxic proteins, impaired axonal transport, inflammation and neurodegeneration. Butanetap inhibits the production of multiple neurotoxic proteins simultaneously.

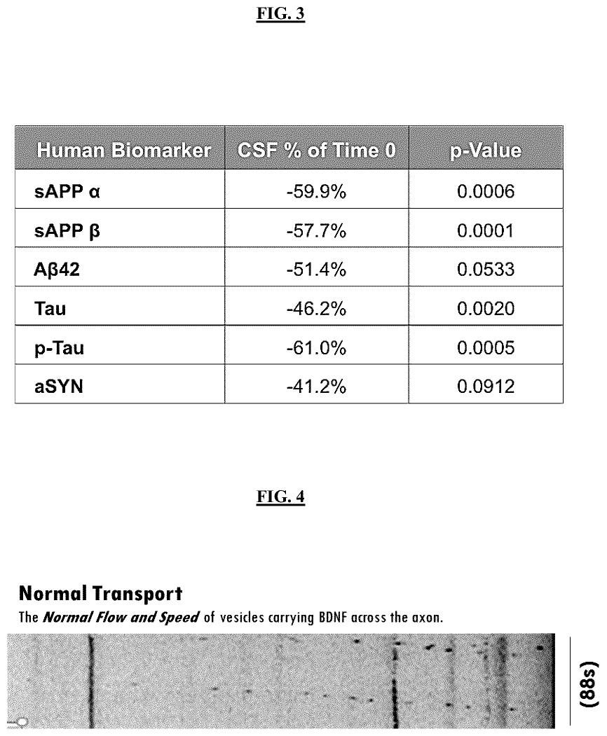

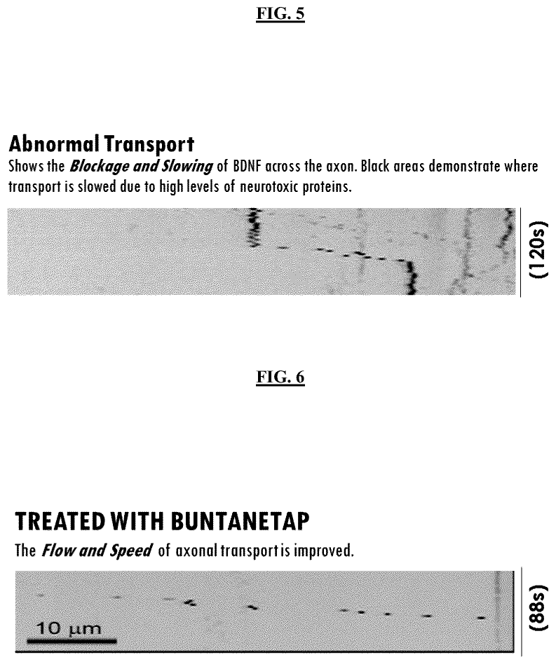

FIG. 4 is a diagram showing normal axonal transport (retrograde 0.5 frame/sec), which depicts the normal flow and speed of vesicles carrying D+BDNF across the axon. FIG. 5 shows abnormal transport, by showing the blockage and slowing of BDNF across the axon. The black areas demonstrate where transport is slowed due to high levels of neurotoxic proteins. Finally, FIG. 6 depicts axonal transport where there is treatment with butanatap. APP, Ab42, C99—Mobley, UCSD; αSYN—Isacson, Harvard; Lee, U. Penn; Tau—U. Muenich & Zuerich; Htt—Mobley, UCSD; TDP43—Taylor, Northwestern. As one can readily see in FIG. 6, butanetap improves the flow and speed of axonal transport. (Chen X Q et al. Alzheimer's & Dementia 08/2020). High levels of neurotoxic proteins, on the other hand, has been shown to provide impaired axonal transport, slower synaptic transmission, inflammation and death of nerve cells, and loss of cognitive and motor function. Butanetap improves dopaminergic neuropathology and working memory in a rate model of traumatic brain injury, and it protects nerve cells from dying the eye of glaucoma rats. By lowering levels of neurotoxic proteins, buntanetap improves axonal transport and impedes the toxic cascade by improving axonal transport, increasing synaptic transmission, decreasing or eliminating inflammation, providing healthy nerve cells, and improving cognitive and motor function.

In Example 5, studies in eight animal and human models showed the following:

| FUNCTION | TEST | SUBJECT |
|---|---|---|
| ANIMALS | | |
| Memory, learning | Mazes | AD mice, DS mice, |
| | | stroke mice, TBI rats |
| Movement | Colonic motility, | PD mice, tau mice |
| | grip strength | |
| Vision | Sight | Glaucoma rats |
| Infections | Cell death | P. Gingivalis mice, |
| | | Covid mice |
| HUMANS | | |
| Cognition, memory, | ADAScog11 * | Early AD patients |
| learning | | |
| Attention, thinking speed | WAIS coding ** | Early AD patients |
| Movement, coordination | MDS-UPDRS *** | Early PD patients |
| Movement speed | WAIS coding **** | Early PD patients |

### Example 6

AD (Alzheimer's disease) and DS (Down syndrome) share several characteristic manifestations. DS is caused by trisomy of whole or part of chromosome 21; this chromosome contains about 233 protein-coding genes, including APP. Recent evidence points to a defining role for increased expression of the gene for APP and for its 99 amino acid C-terminal fragment (C99, also known as 3-CTF) in dysregulating the endosomal/lysosomal system.

The latter is critical for normal cellular function and in neurons for transmitting neurotrophic signals. In Chen X, et al., DOI 10.1002/alz.12185, J. Alzeimer's & Dementia (2020, measures to reduce the levels of fl-APP protein and its products in models of DS will act to prevent or reduce endosomal dysfunction and restore trophic signaling. We begin by detailing our studies on Buntanetap. Buntanetap negatively regulates APP translation.53,58 The proposed mechanism of action of buntanetap builds on a regulatory role for iron in APP expression. The APP 5′-UTR contains an iron-response element (IRE) stem loop that mediates translational control of APP expression.60 The IREs are 30-nucleotide RNA motifs containing the classic 5′-CAGUGX-3′ (X=U, C, or A) sequence. The APP TRE with 5′-CAGAGC motif is homologous with the canonical TRE RNA stem-loop that binds the iron regulatory proteins (IRP1 and IRP2) to control iron-dependent translation.61,62 Among them, IRP1, but not IRP2, binds to the APP IRE.63 IRP binding to the IRE prevents the release of the messenger RNA (mRNA) and, therefore, its association with the ribosome, thus suppressing translation. In the presence of increased cellular iron levels, iron binds to IRP to induce a conformational change leading to dissociation of IRP1 from APP mRNA, thus promoting translation.62,64 Studies of 5′-UTR IRE stem loop in the mRNA of SNCA encoding α-synuclein motivates a model of buntanetap in which the compound increases the affinity of the IRP for the IRE, leading to decreased translation of APP mRNA.54 The result of buntanetap treatment is reduced levels of fl-APP and its products.

In that study, the 5′-UTRs of App mRNAs were amplified from the brains of both 2N and Ts65Dn mice and aligned with the corresponding sequences of mouse and human APP mRNAs from the National Center for Biotechnology Information Entrez data base. The predicted IRE sequences and conserved CAGAGC loop from 2N and Ts65Dn mice were identical with the mouse sequence in the database (Supplementary Figure S1), providing a theoretical basis for manipulation of APP expression by buntanetap in Ts65Dn mice. The author's hypothesis argues that by using buntanetap to normalize fl-APP and its products in DS it will also normalize the structure and function of early endosomes with a reduction in AD-DS relevant phenotypes. This was addressed through studies of buntanetap in a DS mouse model. Therein, the authors showed that buntanetap rescued the deficit in the retrograde axonal transport of BDNF in cortical neurons. (A) Experimental design. Primary cultures of 2N and Ts65Dn cortical neurons were treated at DIV5 with Buntanetap (5 μM) for 48 hours, followed by live imaging. Microfluidic chamber in which primary cultures of 2N and Ts65Dn neurons were maintained; the distal axons of neurons were fluidically isolated. A second series of studies were initiated to study aged Ts65Dn mice using 50 mg/kg/d i.p. for 3 weeks and during behavioral testing for an additional 5 days to examine effects at 16 months, an age at which dysfunction and degeneration are present from neuron cell bodies. QD-BDNF was added to the axonal chamber in preparation for tracking axonal transport of BDNF by live cell imaging. As in vitro, buntanetap treatment of Ts65Dn mice significantly reduced fl-APP. As in vitro, two bands were detected and quantified together. Although no statistically significant difference was detected comparing Ts65Dn and 2N or comparing vehicle with buntanetap treatment, there was an apparent decrease in the intensity of the upper band with buntanetap. Not evident in vitro, this change is unexplained but could reflect a difference in the level of phosphorylated tau species. Consistent with this suggestion, as assessed by immunoblotting for phosphorylation of tau on Thr231 and PHF1 epitopes, the authors discovered that buntanetap reduced phosphorylation of tau in Ts65Dn mice, and that buntanetap-mediated normalization of fl-APP and its products restored normal activity of Rab5 in vivo. The authors also determined that buntanetap treatment restored the levels of pTrkB, pAkt, pERK, and pCREB in Ts65EN brains.

### Example 7

Marble Burying Test described by Broekkamp et al (Eur. J. Pharmacol., 126, 223-229, 1986) was conducted in mice. The test is based on the premise that mice exposed to novel object(s) (i.e., marbles) will bury them in the sawdust floor covering. A decrease in the obsessive-compulsive behavior is evidenced by a decrease the number of marbles buried by mice after administration of a therapeutic agent, as compared to a placebo. This test is sensitive to antidepressant and anxiolytic agents used in the clinic such Benzodiazepines, SSRIs and 5 HT1A compounds.

Mice were individually placed in a test arena (32×21 cm), containing 25 black marbles evenly distributing in 5 rows of 5 on the surface of the bedding, for a 30-minute testing session. The testing protocol and groups used are provided in Table A.

TABLE A

| | | Treatment | Treatment | | | |
|---|---|---|---|---|---|---|
| | Number | p.o. once | p.o. 60 minutes | | | Administration |
| | of | daily (AM) from | before the test | Dose-level | Concentration | Volume |
| Group | animals | Day 1 to Day 9 | on Day 10 | (mg/kg) | (mg/mL) | (mL/kg) |
| 1 | 10 males | Physiological | Physiological | 0 | 0 | 10 |
| | | saline | saline | | | |
| 2 | 10 males | Buntanetap | Buntanetap | 25 | 2.5 | 10 |
| 3 | 10 males | 0.2% HPMC in | Clobazam | 32 | 3.2 | 10 |
| | | distilled water | | | | |

Buntanetap was a therapeutic agent. Physiological saline did not contain any therapeutic agent(s) and was used as a control. Clobazam, a therapeutic agent with confirmed anti obsessive-compulsive activity, was used to validate the test.

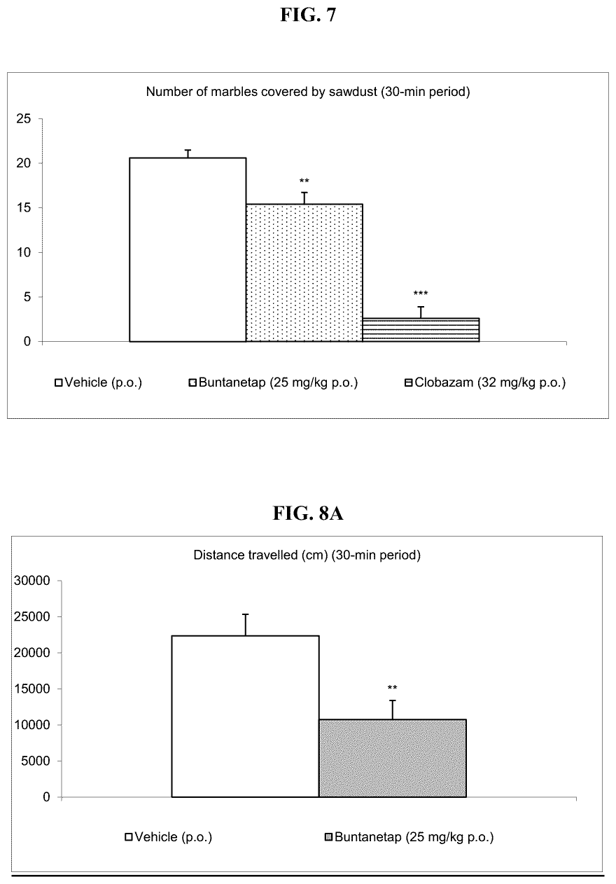

A summary of the number of marbles covered by sawdust for each group is shown in FIG. 7.

Based on the result of the test, it was concluded that, as compared to physiological saline, buntanetap reduces obsessive-compulsive behavior by 20 to 60%, which confirms the anti obsessive-compulsive efficacy of buntanetap.

### Example 8

A number of Elevated Plus-Maze Tests described by Handley and Mithani (Naunyn. Schmied. Arch. Pharmacol., 327, 1-5, 1984) were conducted in mice. The test is based on the premise that mice avoid open spaces (the open arms of an elevated plus-maze). Anxiolytics increase exploratory activity in the open arms, as indicated by increased time spent on the open arms and/or by increased % open-arm entries.

The maze consisted of 4 arms of equal length and width (14×5 cm) arranged in the form of a plus sign (+). Two opposite arms were enclosed by 12 cm high walls (closed arms). The 2 other arms had no walls (open arms). The maze is raised approximately 60 cm above the floor. A mouse was placed in the center of the plus-maze and left to explore for 5 minutes. The testing protocol and groups used in one test are provided in Table B.

TABLE B

| | | Treatment | Treatment | | | |
|---|---|---|---|---|---|---|
| | Number | p.o. once | p.o. 60 minutes | | | Administration |
| | of | daily (AM) from | before the test | Dose-level | Concentration | Volume |
| Group | animals | Day 1 to Day 9 | on Day 10 | (mg/kg) | (mg/mL) | (mL/kg) |
| 4 | 10 males | Physiological | Physiological | 0 | 0 | 10 |
| | | saline | saline | | | |
| 5 | 10 males | Buntanetap | Buntanetap | 25 | 2.5 | 10 |
| 6 | 10 males | 0.2% HPMC in | Clobazam | 16 | 1.6 | 10 |
| | | distilled water | | | | |

Buntanetap was a therapeutic agent. Physiological saline did not contain any therapeutic agent(s) and was used as a control. Clobazam, a therapeutic agent with confirmed anxiolytic activity, was used to validate the test.

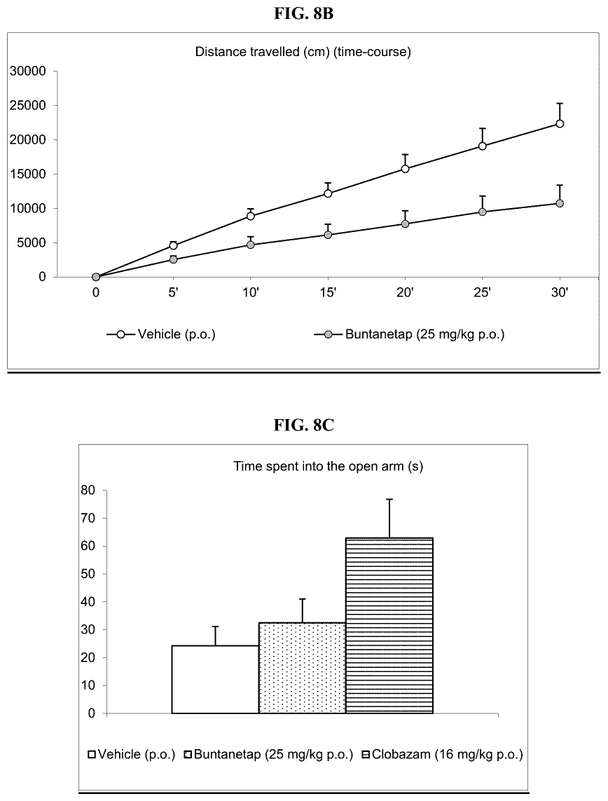

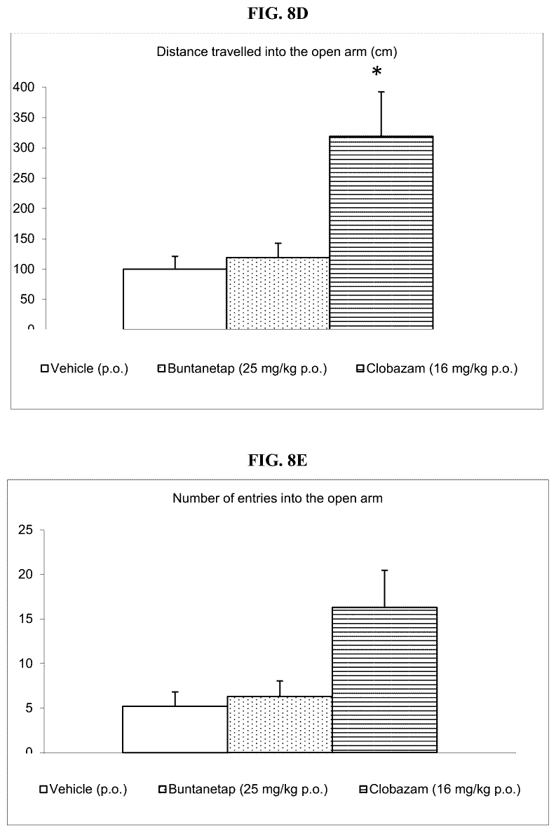

Summaries of the results of the tests are shown in FIG. 8A to 8E. Mice that received buntanetap entered the high open arm of the maze more often, stayed longer and traveled longer distances.

Based on the result of the tests, it was concluded that, as compared to physiological saline, buntanetap lowers the anxiety associated with being in a high maze by 40 to 80%, which confirms the anxiolytic efficacy of buntanetap.

### Example 9

Sensory perception of electric foot shock was examined in different groups of mice through the threshold assessment test. APP/PS1 mice (i.e., the heterozygous double transgenic mice expressing both human APP (K670N:M671L) and human PS1 (M146L)) and WT mice (i.e., the Wild type, a normal healthy mouse with no deficiencies) were used.

The animals were placed in the conditioning chamber and the electric current (0.1 mA for 1 sec) was increased at 30 sec intervals from 0.1 mA to 0.7 mA. Threshold to flinching (first visible response to shock), jumping (first extreme motor response), and vocalized response were quantified for each animal by averaging the shock intensity at which each animal showed the behavioral response to that type of shock. The test is based on the premise that no differences in the threshold assessment among the different groups of mice should be found if treatment with the combination does not affect animals' sensory threshold.

Then, fear conditioning (FC) training was performed. Mice was placed in a conditioning chamber for 2 min before the onset of a tone (Conditioned Stimulus (CS), 30 sec, 85 dB sound at 2800 Hz). In the last 2 sec of the CS, mice was given a 2 sec, 0.7 mA mild foot shock (Unconditioned Stimulus, (US)) through the bars of the floor. After the US, the mice was left in the chamber for another 30 sec. Freezing behavior, defined as the absence of movements except for that needed for breathing, was scored using Freezeview software (Med Associates, St. Albans, VT). Contextual fear learning, a type of memory for which hippocampal function is indispensable, was evaluated 24 hrs after training by measuring freezing responses for 5 min in the same chamber where the mice were trained. Cued fear learning, a type of memory that depends on amygdala function, was evaluated 24 hrs after contextual testing. The mice was placed in a novel context for 2 min (pre-CS test), after which they were given a CS for 3 min (CS test), and freezing behavior was measured during the first 30 sec that mimic the CS-US conditioning and the remaining 2.5 min. A decrease in % freezing between the shocked and unshocked animals is evidence of fear-reducing efficacy.

The tested groups were as follows:
1. APP/PS1+Vehicle;
2. APP/PS1+Buntanetap (1 mg/Kg);
3. APP/PS1+Buntanetap (10 mg/Kg); and
4. WT+Vehicle.

Mice that received buntanetap had less fear than mice that did not.

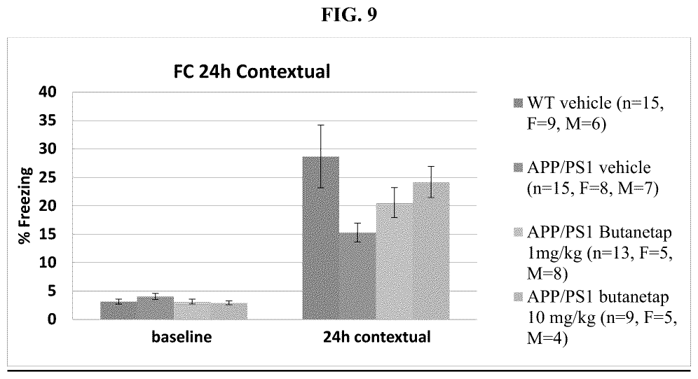

Summaries of the results are shown in FIG. 9.

Based on the result of the tests, it was concluded that, as compared to WT vehicle, buntanetap lowers the fear response to having learned that they receive an electric shock in the dark box by 40 to 90%, which confirms the fear-reducing efficacy of buntanetap.

While the invention has been disclosed with reference to specific embodiments, it is apparent that other embodiments and variations of this invention may be devised by others skilled in the art without departing from the true spirit and scope of the invention. All patents and publications cited herein are incorporated by reference in their entirety. The appended claims are intended to be construed to include all such embodiments and equivalent variations.

What is claimed is:

1. A method for treating, inhibiting, reducing, slowing, or delaying symptoms of a mental illness comprising administering to an animal a therapeutically effective amount of a compound selected from the group consisting of Formula (I), Formula (II) and Formula (III): 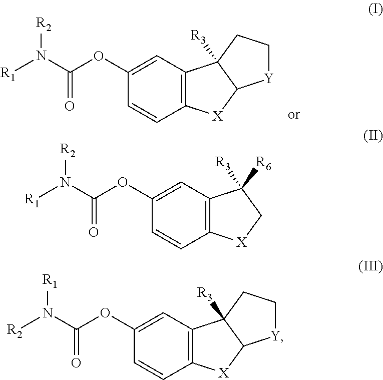
wherein
in Formula (I) and Formula (II), R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein, in Formula (III), R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl; and
wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof.

2. The method of claim 1, wherein the compound of Formula (I) or Formula (II) is the substantially pure (+)-enantiomer.

3. The method of claim 1, wherein the compound of Formula (III) is the substantially pure (−)-enantiomer.

4. The method of claim 1, wherein the compound is buntanetap of Formula (IV), 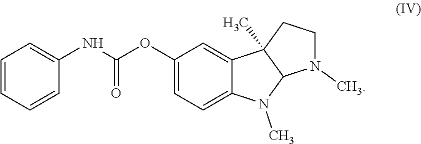 [SMILES: CN1CC[C@]2(C)c3cc(OC(=O)Nc4ccccc4)ccc3N(C)C12]
wherein the compound of Formula (IV) is the substantially pure (+)-enantiomer, or a pharmaceutically acceptable salt thereof.

5. The method of claim 1, wherein the compound is administered via a route selected from the group consisting of orally, parenterally, sublingually, via suppository, nasally, topically, transdermally, and via implant under the skin.

6. The method of claim 1, wherein the compound is chronically administered to the human who is experiencing a mental illness selected from the group consisting of autism, attention deficit-hyperactivity disorder, bipolar disorder, major depressive disorder, and schizophrenia.

7. The method of claim 4, wherein buntanetap or a pharmaceutically acceptable salt thereof is administered (i) orally in an amount from about 1 mg to about 120 mg on a once-a-day basis; (ii) intravenously in an amount from about 0.1 mg to about 25 mg/day; or (ii) intraperitoneally/intramuscularly (IP/IM) in a dose from about 0.3 to about 70 mg/day.

8. The method of claim 7, wherein buntanetap or a pharmaceutically acceptable salt thereof is administered orally in an amount from about 10 mg to about 80 mg on a once-a-day basis.

9. The method of claim 7, wherein peak plasma circulating levels of buntanetap in humans range from about 1 ng/mL to about 380 ng/mL.

10. The method of claim 1, wherein the compound is administered in a pharmaceutical formulation together with one or more pharmaceutically acceptable excipients.

11. The method of claim 6, wherein compound is buntanetap.

12. A neuropsychotic pharmaceutical composition, comprising:
(i) a therapeutically effective amount of a compound selected from the group consisting of Formula (I), Formula (II) and Formula (III): 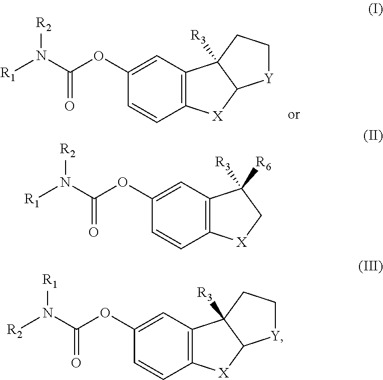
wherein,
in Formula (I) and Formula (II), R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III), R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl; and
wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof;
(ii) an effective amount of compound selected from the group consisting of an antipsychotic agent, an antidepressant agent, a hallucinogenic agent, and pharmaceutically acceptable salts thereof; and at least one pharmaceutically acceptable excipient.

13. The pharmaceutical composition of claim 12, wherein the compound of Formula (III) is the substantially pure (−)-enantiomer.

14. The pharmaceutical composition of claim 12, wherein the compound is buntanetap of Formula (IV), 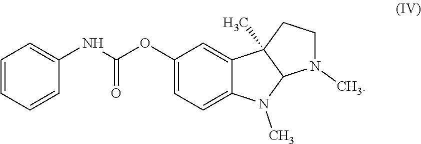 [SMILES: CN1CC[C@]2(C)c3cc(OC(=O)Nc4ccccc4)ccc3N(C)C12]
wherein the compound of Formula (IV) is the substantially pure (+)-enantiomer, or a pharmaceutically acceptable salt thereof.

15. The pharmaceutical composition of claim 14, wherein the buntanetap or a pharmaceutically acceptable salt thereof is in an amount from about 1 mg to about 120 mg.

16. The pharmaceutical composition of claim 14, wherein buntanetap or a pharmaceutically acceptable salt thereof is in an amount from about 10 mg to about 120 mg.

17. The pharmaceutical composition of claim 12, wherein the antipsychotic agent is a therapeutically effective amount of a typical antipsychotic agent, an atypical antipsychotic agent, a miscellaneous antipsychotic agent, pharmaceutically acceptable salts thereof, and combinations of any of the foregoing.

18. The pharmaceutical composition of claim 12, wherein the antipsychotic agent, the antidepressant agent or the hallucinogenic agent is included in a subtherapeutic amount.

19. The pharmaceutical composition of claim 12, wherein the antipsychotic agent is selected from the group consisting of risperidone, quetiapine, olanzapine, ziprasidone, paliperidone, aripiprazole, clozapine, haloperidol, pimozide, molidone, loxapine, prochlorperazine, chlorpromazine, perphenazine, fluphenazine, thioridazine, mesoridazine, thiothixene, asenapine, benperidol, zuclopenthixol, flupentixol, fluphenazine, lurasidone, levomepromazine, promazine, pericyazine, caripraine, amisulpride, trifluorperazine, sulpiride, acepromazine, acetophenazine, ziprasidone, iloperidone, pharmaceutically acceptable salts thereof, and combinations of any of the foregoing.

20. The pharmaceutical composition of claim 12, which is an oral dosage form.

21. The pharmaceutical composition of claim 12, wherein the amount of the compound of formula (I), (II), and (III), or pharmaceutically acceptable salts thereof and the additional therapeutic agent selected from an antipsychotic agent, an antidepressant agent, and a hallucinogenic agent is effective to treating, inhibiting, reducing, slowing or delaying the symptoms of a mental illness.

22. The pharmaceutical composition of claim 21, wherein the mental illness is selected from the group consisting of autism, attention deficit-hyperactivity disorder, bipolar disorder, major depressive disorder, and schizophrenia.

23. The method of claim 1, wherein the mental illness presents as a disruption in the genetic code of the human genetic code disruption in CACNA1C or CACNB2.

24. The method of claim 1, further comprising co-administering an effective amount of compound selected from the group consisting of an antipsychotic agent, an antidepressant agent, a hallucinogenic agent, and pharmaceutically acceptable salts thereof.
